(19) United States
(12) Patent Application Publication
(10) Pub. No.: US 2025/0236865 A1
(43) Pub. Date: Jul. 24, 2025

(54) METHODS FOR CAPTURING LIBRARY DNA FOR SEQUENCING

(71) Applicant: Illumina, Inc., San Diego, CA (US)

(72) Inventors: James Tsay, San Diego, CA (US); Lewis J. Kraft, San Diego, CA (US); Mathieu Lessard-Viger, San Diego, CA (US); Jeffrey D. Brodin, San Diego, CA (US); Rohit Subramanian, San Diego, CA (US); Eric M. Brustad, San Diego, CA (US)

(21) Appl. No.: 18/699,869

(22) PCT Filed: Oct. 18, 2022
(86) PCT No.: PCT/US2022/078267

§ 371 Date: Apr. 9, 2024

Related U.S. Application Data
(60) Provisional application No. 63/257,981, filed on Oct. 20, 2021.

(51) Int. Cl. C12N 15/10; C12Q 1/6874

(52) U.S. Cl. CPC C12N 15/1093; C12Q 1/6874

(57) ABSTRACT

Embodiments of the present disclosure relate to methods of capturing library DNA complexes to the patterned surface of the solid support for sequencing. The methods described herein improve the monoclonality of clusters and sequencing data quality and read length.

### FIELD

The present disclosure relates to methods of capturing library DNA on a surface of a solid support for sequencing applications, such as nucleic acid sequencing.

### REFERENCE TO SEQUENCE LISTING

The present application is being filed along with a Sequence Listing in electronic format. The Sequence Listing is provided as a file entitled Sequence_listing_ILLINC.567WO.xml created Oct. 17, 2022, which is 21.7 KB in size. The information in the electronic format of the Sequence Listing is incorporated herein by reference in its entirety.

### BACKGROUND

Many current sequencing platforms use “sequencing by synthesis” (SBS) technology and fluorescence-based methods for detection. In some examples, numerous target polynucleotides isolated from a library to be sequences, or template polynucleotides, are attached to a surface of a substrate in a process known as seeding. Multiple copies of the template polynucleotides may then be synthesized in attachment to the surface in proximity to where a template polynucleotide of which it is a copy was seeded, in a process called clustering. Subsequently, nascent copies of the clustered polynucleotides are synthesized under conditions in which they emit a signal identifying each nucleotide as it is attached to the nascent strand. Clustering of a plurality of copies of the seeded template polynucleotide in proximity to where it was initially seeded results in amplification of signal generated during the visualizable polymerization, improving detection.

Seeding and clustering for SBS work well when as much of an available substrate surface as possible is seeded by template polynucleotides, which may maximize an amount of sequencing information obtainable during a sequencing run. In general, the less available surface area of a substrate used for seeding and clustering, the less efficient an SBS process may be, resulting in increased time, reactants, expense, and complicated data processing for obtaining a given amount of sequencing information of a given library.

A library of template polynucleotides (library DNA) may generally include a high number of template polynucleotide molecules whose nucleotide sequences differ from each other's. If two such template polynucleotides seed too closely together on a surface of a substrate (for example, an unpatterned surface), clustering may result in spatially comingled populations of copied polynucleotides, some of which having a sequence of one of the template polynucleotides that seeded nearby and others having a sequence of another template polynucleotide that also seeded nearby on the surface. Or two clusters formed from two different template polynucleotides that seeded in too close proximity to each other may be too adjacent to each other or adjoin each other such that an imaging system used in an SBS process may be unable to distinguish them as separate clusters even though there may be no or minimal spatial comingling of substrate-attached sequences between the clusters. Such a disadvantageous condition may generally be referred to as polyclonality. For a patterned surface containing a plurality of confined compartments or location (such as a surface containing a plurality of nanowells separated by interstitial regions), polyclonality generally results from multiple seeding of different template polynucleotides in the same confined location and the subsequent amplification process produce more than comingled populations of copied of template polynucleotides in the same confined location. Seeding and clustering also work well when template polynucleotides from a library with different sequences seed on, or attach to, positions of the surface (e.g., an unpatterned surface) sufficiently distal from each other such that clustering results in spatially distinct clusters of copied polynucleotides each resulting from the seeding of a single template polynucleotide, a condition generally referred to as monoclonality. For a patterned surface, monoclonality refers to the condition when each compartment or confined area (e.g., nanowell) is seeded with a single template polynucleotide, or a single dominant template polynucleotide, such that clustering results in a single cluster of identical copies of the same template polynucleotide or a single dominant cluster in the same compartment or confined location. Polyclonality may result in lower library usage, higher noise to signal ratio during sequencing, and lower data quality. Therefore, it is desirable to perform SBS under conditions under which as much available surface area as possible of a substrate surface is used for seeding and clustering, while also promoting separation of seeded template polynucleotides so as to maximize monoclonality of clusters as possible and minimize polyclonal clusters. As such, there exist a need to develop new methods for improving monoclonality of the polynucleotides during the clustering process.

### SUMMARY

Some aspect of the present disclosure relates to a solid support for use in sequencing, having a patterned surface comprising a plurality of library DNA binding regions separated by interstitial regions;

wherein each library DNA binding region comprises a number of capture moieties adapted for capturing a library DNA complex, and at least a portion of the interstitial regions comprise clustering oligonucleotides; and wherein the capture moieties are orthogonal to the clustering oligonucleotides, and the size, dimension, or diameter of each library binding regions is from about 10 nm to about 100 nm, or from about 20 nm to about 50 nm.

In some embodiments of the solid support described herein, the patterned surface comprises a plurality of nanowells, and at least a portion of the nanowells each comprises one library DNA binding region that is inside the nanowell. In some other embodiments, each of the library DNA binding region is a nanowell forming a patterned surface of capture sites, or a capture pad on the surface (e.g., an unpatterned surface) forming a planar array of capture sites.

Some additional aspect of the present disclosure relates to a solid support for use in sequencing, having a patterned surface comprising a plurality of library DNA binding regions separated by interstitial regions;

wherein each library DNA binding region comprises a number of capture moieties adapted for capturing a library DNA complex, and at least a portion of the interstitial regions comprise clustering oligonucleotides;

wherein each library DNA complex comprises a single library polynucleotide and a number of accessory binding sites capable of binding to the capture moieties in the library DNA binding region by noncovalent or covalent interactions;

wherein the number of accessory binding sites on each library DNA complex is more than the number of capture moieties in each library DNA binding region; and

wherein the capture moieties are orthogonal to the clustering oligonucleotides.

In some embodiments of the solid support described herein, the solid support further comprises a plurality of library DNA complexes attached to the library binding regions through the capture moieties, wherein at least 50%, 55%, 60%, 65%, 70%, 75%, 80%, 85%, 90%, 95%, 98%, or 99% of the library DNA binding regions are each occupied with no more than three library DNA complexes. In further embodiments, at least 50%, 55%, 60%, 65%, 70%, 75%, 80%, 85%, 90%, 95%, 98%, or 99% of the library DNA binding regions are each occupied with a single library DNA complex or only a single dominant library DNA complex. In some further embodiments, amplification of the library DNA complex in at least 50%, 55%, 60%, 65%, 70%, 75%, 80%, 85%, 90%, 95%, 98%, or 99% of the DNA binding regions results in a monoclonal cluster or a single dominant cluster. In some embodiments, at least 50%, 55%, 60%, 65%, 70%, 75%, 80%, 85%, 90%, 95%, 98%, or 99% of clusters on the solid support generated from the amplification are monoclonal clusters.

In some embodiments of the solid support described herein, each library DNA complex comprises a scaffold, wherein the number of accessory binding sites are on the scaffold, and the library polynucleotide is attached to the scaffold by non-covalent or covalent interaction with a single template binding site on the scaffold. In some such embodiments, the scaffold comprises a nanoparticle, a dendrimer, a polymer with bottlebrush structure, a single strand DNA scaffold, or a polypeptide scaffold, or a combination thereof. In further embodiments, the scaffold comprises a DNA dendrimer, a single strand DNA with bottlebrush structure, or a single strand DNA produced by rolling circle amplification (RCA).

In some embodiments of the solid support described herein, the library polynucleotide is attached to the scaffold by noncovalent hybridization with the single template binding site. In other embodiments, the library polynucleotide is attached to the scaffold by covalent bonding with the single template binding site. In some such embodiments, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

In some embodiments of the solid support described herein, at least a portion of the capture moieties are adapted to capture the library DNA complex by non-covalent interactions with one or more accessory binding sites on the library DNA complex. In some such embodiments, at least a portion of the capture moieties each comprises an oligonucleotide seeding sequence that is capable of hybridizing with one accessory binding site on the library DNA complex. In further embodiments, the oligonucleotide seeding sequence comprises from about 5 to about 50 nucleotides, from about 10 to about 40 nucleotides, or from about 20 to about 30 nucleotides. In other embodiments, at least a portion of the capture moieties are adapted to capture the library DNA complex by avidin (e.g., streptavidin) biotin interaction.

In some other embodiments of the solid support described herein, at least a portion of the capture moieties are adapted to capture the library DNA complex by forming covalent bonding with accessory binding sites on the library DNA complex. In some such embodiments, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

In some embodiments of the solid support described herein, the solid support is a flowcell, containing a plurality of nanowells on the patterned surface of the flowcell. In some other embodiments, the solid support contains a plurality of capture pads, forming a patterned surface on the solid support. In some further embodiments, at least a portion of the nanowells each comprises a single library DNA binding region that is inside the nanowell. In other embodiments, each library DNA binding region is a nanowell or a capture pad, forming a patterned surface in the flowcell. In some embodiments, the size, dimension or diameter of each library binding region is from about 10 nm to about 100 nm, or about 20 nm to about 50 nm.

In some embodiments of the solid support described herein, at least a portion of the library DNA binding regions each comprises from about 1 to about 200, from about 10 to about 100, or from about 20 to about 50 capture moieties. In some embodiments, the ratio of the clustering oligonucleotides to the capture moieties is from about 10 to 100,000.

In some embodiments of the solid support described herein, the clustering oligonucleotides comprise P5 and P7 primers, or P15 and P17 primers.

Some additional aspect of the present disclosure relates a method of preparing a patterned surface of a solid support for sequencing, comprising:
contacting a solution comprising a plurality of library DNA complexes with the patterned surface comprising a plurality of library DNA binding regions separated by interstitial regions;
   wherein each library DNA binding region comprises a number of capture moieties adapted for capturing one library DNA complex, at least a portion of the interstitial regions comprise clustering oligonucleotides, and the capture moieties are orthogonal to the clustering oligonucleotides;
   wherein each library DNA complex comprises a single library polynucleotide and a number of accessory binding sites, and the number of accessory binding sites on each library DNA complex is more than the number of capture moieties in each library DNA binding region; and
attaching the plurality of library DNA complexes to the library DNA binding regions of the pattered surface by noncovalent or covalent interactions between the accessory binding sites of the library DNA complexes and the capture moieties of library DNA binding regions.

In some embodiments of the method described herein, wherein at least 50%, 55%, 60%, 65%, 70%, 75%, 80%, 85%, 90%, 95%, 98%, or 99% of the library DNA binding regions are each occupied with no more than three library DNA complexes. In further embodiments, at least 50%, 55%, 60%, 65%, 70%, 75%, 80%, 85%, 90%, 95%, 98%, or 99% of the library DNA binding regions are each occupied with a single library DNA complex or only a single dominant library DNA complex.

In some embodiments, the method further comprises amplifying the library polynucleotides. In some such embodiments, amplification of the library DNA complex in at least 50%, 55%, 60%, 65%, 70%, 75%, 80%, 85%, 90%, 95%, 98%, or 99% of the DNA binding regions results in a monoclonal cluster or a single dominant cluster. In some further embodiments, at least 50%, 55%, 60%, 65%, 70%, 75%, 80%, 85%, 90%, 95%, 98%, or 99% of the clusters generated from the amplification are monoclonal clusters.

In some embodiments of the method described herein, the solid support comprises a flowcell, for example a patterned flowcell. In other embodiments, the solid support surface such as flowcell surface may be unpatterned. In some such embodiments, the patterned flowcell surface containing a plurality of nanowells, at least a portion of the nanowells each comprises a single library DNA binding region that is inside the nanowell. In other embodiments, each of the library DNA binding region is a nanowell or a capture pad, forming a patterned surface on the flowcell. In further embodiments, the size, dimension or diameter of each library binding regions is from about 10 nm to about 100 nm, or about 20 nm to about 50 nm.

In some embodiments of the method described herein, each library DNA complex comprises a scaffold, wherein the number of accessory binding sites are on the scaffold, and the single library polynucleotide is attached to the scaffold by non-covalent or covalent interaction with a single template binding site on the scaffold. In some such embodiments, the scaffold comprises a dendrimer, a polymer with bottlebrush structure, a single strand DNA scaffold, or a polypeptide scaffold. In some embodiments, the library polynucleotide is attached to the scaffold by noncovalent hybridization with the single template binding site. In other embodiments, the library polynucleotide is attached to the scaffold by covalent bonding with the single template binding site. In some such embodiments, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

In some embodiments of the method described herein, at least a portion of the capture moieties each comprises an oligonucleotide seeding sequence, and each library DNA complex is attached to the library DNA binding region by hybridization of one or more accessory binding sites on the library DNA complex with the oligonucleotide seeding sequences. In some such embodiments, the oligonucleotide seeding sequence comprises from about 10 to about 40 nucleotides, or from about 20 to about 30 nucleotides. In other embodiments, at least a portion of the capture moieties each comprises an avidin moiety, at least a portion of the accessory binding sites on the library DNA complex each comprises a biotin moiety, and the library DNA complex is attached to the library DNA binding region by avidin (e.g., streptavidin) biotin interaction. In some other embodiments, each library DNA complex is attached to the library DNA binding region by covalent bonding. The covalent bonding may include but not limited to amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

In some embodiments of the method described herein, at least a portion of the library DNA binding regions each comprises from about 1 to about 200, from about 10 to about 100, or from about 20 to about 50 capture moieties. In some embodiments, the ratio of the clustering oligonucleotides to the capture moieties is from about 10 to 100,000.

In some embodiments of the method described herein, the clustering oligonucleotides comprise P5 and P7 primers, or P15 and P17 primers as described herein.

Additional aspect of the present disclosure relates to a DNA library comprising a plurality of library DNA complexes, each library DNA complex comprises a single library polynucleotide, and a scaffold comprising a number of accessory binding sites adapted for attaching to a number of capture moieties on a library DNA binding region of a patterned solid support,
wherein the library polynucleotide comprises an insert and an adaptor region;
wherein the adaptor region comprises a clustering sequence, each accessory binding sites comprises a complementary capture moiety, wherein the clustering sequence and the complementary capture moiety are orthogonal; and
wherein the number of accessory binding sites on each library DNA complex is more than the number of capture moieties in the library DNA binding region.

In some embodiments of the DNA library described herein, the library DNA complex further comprises a spacer region between the clustering sequence and the scaffold. In some such embodiments, the spacer region comprises a linker. In further embodiments, the linker comprises a PEG linker.

In some embodiments of the DNA library described herein, the library polynucleotide further comprises an index sequence (e.g. i5), a first sequencing binding site (e.g. SBS3), a second sequencing binding site (e.g. SBS12), and/or a second index sequence (e.g. i7).

In some embodiments of the DNA library described herein, the adaptor region comprises a P5, P7, P15 or P17 sequence, or a sequence that is complementary or at least partially complementary to the P5, P7, P15 or P17 sequence (i.e., P5′, P7′, P15′ or P17′ sequence).

In some embodiments of the DNA library described herein, the DNA library is a double-stranded library. In other embodiments, the DNA library is a single-stranded library.

In some embodiments of the DNA library described herein, the scaffold comprises a DNA dendrimer, a polymer with bottlebrush structure, a single strand DNA with bottlebrush structure, a single strand DNA produced by rolling circle amplification, or a polypeptide scaffold, or a combination thereof. In some further embodiments, each complementary capture moiety of the DNA complex comprises an oligo sequence that is hybridizable to the capture moiety on the library binding region of the patterned solid support.

In any embodiments of method, the solid support or the DNA library described herein, each of the capture moieties of the DNA biding regions may comprise an oligo sequence that is orthogonal to the clustering oligos. The capture moiety is also referred to as a seeding primer or seeding oligo.

### BRIEF DESCRIPTION OF THE DRAWINGS

FIG. 1 illustrates an example of an orthogonal seeding strategy by incorporating a complementary seeding oligo (PX′) to a standard adaptor sequence of a library DNA via a linker.

FIG. 2 illustrates an embodiment of a library DNA seeding method described herein by incorporating a number of complementary seeding oligos (PX′) to a standard adaptor sequence of a library DNA via a linker and/or a scaffold.

FIG. 3 is an amplified view of a library DNA complex comprising a number of complementary seeding oligos (PX′) hybridized to an DNA binding region of a solid support according to an embodiment of the present disclosure.

FIGS. 4A-4C each illustrates a cross-section view of a solid support comprising a DNA binding region that is discrete or separated by the interstitial regions, according to an embodiment of the present disclosure.

### DETAILED DESCRIPTION

The present disclosure relates to solid support configurations and methods for increasing monoclonal clustering during sequencing-by-synthesis (SBS). The solid support comprises a plurality of library DNA binding regions separated by interstitial regions. Each of the DNA binding regions contains a number of capture moieties adapted for capturing a library DNA complex, and at least a portion of the interstitial regions comprise clustering oligonucleotides. The capture moieties are orthogonal to the clustering oligonucleotides. Each library DNA complex comprises a single library polynucleotide (either single-stranded or double-stranded) and a number of accessory binding sites capable of binding to the capture moieties in the library DNA binding region by noncovalent or covalent interactions, and the number of accessory binding sites on each library DNA complex is more than the number of capture moieties in each library DNA binding region. When a first library DNA (also referred to as a template DNA, library strand, or template strand) is attached to a particular DNA binding region, the capturing moieties in such DNA biding region are bounded to the accessory binding sites of the first library DNA complex. It excludes the binding of any addition or subsequent template DNAs to the same DNA binding region. As a result, any secondary seeding events in the same DNA binding region are disfavored. The solid support and methods described herein improve the monoclonality of the library strand occupied nanowells. In addition, the methods also improve the occupancy of the nanowells on the solid support, signal intensity, and sequencing data quality, as well as the efficiency of the library capture.

The section headings used herein are for organizational purposes only and are not to be construed as limiting the subject matter described.

### Definition

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, common abbreviations are defined as follows:
dATP Deoxyadenosine triphosphate
dCTP Deoxycytidine triphosphate
dGTP Deoxyguanosine triphosphate
dTTP Deoxythymidine triphosphate
PAZAM Poly(N-(5-azidoacetamidylpentyl) acrylamide-co-acrylamide) of any acrylamide to Azapa ratio
SBS Sequencing-by-synthesis

As used herein, the term “attached” refers to the state of two things being joined, fastened, adhered, connected or bound to each other. For example, an analyte, such as a nucleic acid, can be attached to a material, such as a gel or solid support, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “array” refers to a population of different probes (e.g., probe molecules) that are attached to one or more substrates such that the different probes can be differentiated from each other according to relative location. An array can include different probes that are each located at a different addressable location on a substrate. Alternatively or additionally, an array can include separate substrates each bearing a different probe, wherein the different probes can be identified according to the locations of the substrates on a surface to which the substrates are attached or according to the locations of the substrates in a liquid. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached hydrogel refers to a hydrogel that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the term “reversible covalent bond” refers to a covalent bond that can be cleaved for example under the application of heat, light or other (bio) chemical methods (e.g. by exposure to a degradation agent, such as an enzyme or a catalyst), while a “non-reversible covalent bond” is stable to degradation under such conditions. Non-limiting examples of reversible covalent bonds include thermally or photolytically cleavable cycloadducts (e.g. furan-maleimide cycloadducts), alkenylene linkages, esters, amides, acetals, hemiaminal ethers, aminals, imines, hydrazones, polysulfide linkages (e.g. disulfide linkages), boron-based linkages (e.g. boronic and borinic acids/esters), silicon-based linkages (e.g. silyl ether, siloxane), and phosphorus-based linkages (e.g. phosphite, phosphate) linkages.

As used herein, the term “non-covalent interactions” differs from a covalent bond in that it does not involve the sharing of electrons, but rather involves more dispersed variations of electromagnetic interactions between molecules or within a molecule. Non-covalent interactions can be generally classified into four categories, electrostatic, π-effects, van der Waals forces, and hydrophobic effects. Non-limiting examples of electrostatic interactions include ionic interactions, hydrogen bonding (a specific type of dipole-dipole interaction), halogen bonding, etc. Van der Walls forces are a subset of electrostatic interaction involving permanent or induced dipoles or multipoles. π-effects can be broken down into numerous categories, including (but not limited to) T-T interactions, cation-π & anion-π interactions, and polar-π interactions. In general, I-effects are associated with the interactions of molecules with the π-orbitals of a molecular system, such as benzene. The hydrophobic effect is the tendency of nonpolar substances to aggregate in aqueous solution and exclude water molecules. Non-covalent interactions can be both intermolecular and intramolecular. Non-covalent interactions can be both intermolecular and intramolecular.

As used herein, the term “host-guest interaction” refers to two or more groups which are able to form bound complexes via one or more types of non-covalent interactions by molecular recognition, such as ionic bonding, hydrogen bonding, hydrophobic interactions, van der Waals interactions and π-π interactions. For example, the host-guest interaction may include interactions formed between cucubiturils with adamantanes (e.g. 1-adamantylamine), ammonium ions (e.g. amino acids), ferrocenes; cyclodextrins with adamantanes (e.g. 1-adamantylamine), ammonium ions (e.g. amino acids), ferrocenes, calixarenes with adamantanes (e.g. 1-adamantylamine), ammonium ions (e.g. amino acids), ferrocenes; crown ethers (e.g. 18-crown-6, 15-crown-5, 12-crown-4) or cryptands (e.g. [2.2.2]cryptand) with cations (e.g. metal cations, ammonium ions); avidins (e.g. streptavidin) and biotin; and antibodies and haptens.

As used herein, the term “ionic bond” refers to a chemical bond between two or more ions that involves an electrostatic attraction between a cation and an anion. For example, the cation may be selected from “metal cations”, as described herein, or “non-metal cations”. Non-metal cations may include ammonium salts (e.g. alkylammonium salts) or phosphonium salts (e.g. alkylphosphonium salts). The anion may be selected from phosphates, thiophosphates, phosphonates, thiophosphonates, phosphinates, thiophosphinates, sulfates, sulfonates, sulfites, sulfinates, carbonates, carboxylates, alkoxides, phenolates and thiophenolates.

As used herein, the term “hydrogen bond” refers to a bonding interaction between a lone pair on an electron-rich atom (e.g. nitrogen, oxygen or fluorine) and a hydrogen atom attached to an electronegative atom (e.g. nitrogen or oxygen).

As used herein, the term “host-guest interaction” refers to two or more groups which are able to form bound complexes via one or more types of non-covalent interactions by molecular recognition, such as ionic bonding, hydrogen bonding, hydrophobic interactions, van der Waals interactions and π-π interactions. For example, the host-guest interaction may include interactions formed between cucubiturils with adamantanes (e.g. 1-adamantylamine), ammonium ions (e.g. amino acids), ferrocenes; cyclodextrins with adamantanes (e.g. 1-adamantylamine), ammonium ions (e.g. amino acids), ferrocenes, calixarenes with adamantanes (e.g. 1-adamantylamine), ammonium ions (e.g. amino acids), ferrocenes; crown ethers (e.g. 18-crown-6, 15-crown-5, 12-crown-4) or cryptands (e.g. [2.2.2]cryptand) with cations (e.g. metal cations, ammonium ions); avidins (e.g. streptavidin) and biotin; and antibodies and haptens.

As used herein, the term “percent passing filter” or “% PF” is a measure of the ability of a nanowell to be successfully ‘read’ during sequencing. As the grafting density increases, there is an initial increase in % PF which is followed by a rapid decline, due to increased polyclonality within a well leading to a reduction in a clean readable target signal. In other words, as the primer density increases, the likelihood of two or more templates hybridizing onto the surface of the well increases. The presence of more than one template increases the likelihood of both templates being amplified leading to polyclonality and an increased likelihood that the signal strength is reduced or not readable. % PF of the occupied wells can therefore be used to measure the degree of clonality. While reference above is made to nanowells, the same concept is applicable to any solid support or substrate.

As used herein, the term “coat,” when used as a verb, is intended to mean providing a layer or covering on a surface. At least a portion of the surface can be provided with a layer or cover. In some cases, the entire surface can be provided with a layer or cover. In alternative cases only a portion of the surface will be provided with a layer or covering. The term “coat,” when used to describe the relationship between a surface and a material, is intended to mean that the material is present as a layer or cover on the surface. The material can seal the surface, for example, preventing contact of liquid or gas with the surface. However, the material need not form a seal. For example, the material can be porous to liquid, gas, or one or more components carried in a liquid or gas. Exemplary materials that can coat a surface include, but are not limited to, a gel, polymer, organic polymer, liquid, metal, a second surface, plastic, silica, or gas.

As used herein the term “analyte” is intended to include any of a variety of analytes that are to be detected, characterized, modified, synthesized, or the like. Exemplary analytes include, but are not limited to, nucleic acids (e.g., DNA, RNA or analogs thereof), proteins, polysaccharides, cells, nuclei, cellular organelles, antibodies, epitopes, receptors, ligands, enzymes (e g kinases, phosphatases or polymerases), peptides, small molecule drug candidates, or the like. An array can include multiple different species from a library of analytes. For example, the species can be different antibodies from an antibody library, nucleic acids having different sequences from a library of nucleic acids, proteins having different structure and/or function from a library of proteins, drug candidates from a combinatorial library of small molecules, etc.

As used herein the term “contour” is intended to mean a localized variation in the shape of a surface. Exemplary contours include, but are not limited to, wells, pits, channels, posts, pillars, and ridges. Contours can occur as any of a variety of depressions in a surface or projections from a surface. All or part of a contour can serve as a feature in an array. For example, a part of a contour that occurs in a particular plane of a solid support can serve as a feature in that particular plane. In some embodiments, contours are provided in a regular or repeating pattern on a surface.

Where a material is “within” a contour, it is located in the space of the contour. For example, for a well, the material is inside the well, and for a pillar or post, the material covers the contour that extends above the plane of the surface.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules. The term can be similarly applied to proteins which are distinguishable as different from each other based on amino acid sequence differences.

As use herein, the term “one cluster of template polynucleotides” or “one cluster of library DNA” refer to a plurality of identical template polynucleotides immobilized on a particular confined location or compartment of a substrate (e.g., within a single nanowell) as a result of amplification of a single template polynucleotide captured at the particular confined location or compartment (e.g., within the same nanowell) of the substrate. The term “one dominant cluster” or “one dominant cluster of library DNA” is used in the context of polyclonality as described herein, when clustering result in two or more clusters formed from two or more different template polynucleotides that are seeded in the same confined location or compartment (e.g., within the same nanowell). When an imaging system used in an SBS process may be able distinguish them as separate clusters and the cluster that is responsible for the base calling in sequencing is referred to as “the dominant cluster.” The library DNA complex (template polynucleotide) that forms the dominant cluster is referred to herein as “the dominant library DNA complex.”

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “feature” means a location in an array that is configured to attach a particular analyte. For example, a feature can be all or part of a contour on a surface. A feature can contain only a single analyte, or it can contain a population of several analytes, optionally the several analytes can be the same species. In some embodiments, features are present on a solid support prior to attaching an analyte. In other embodiments the feature is created by attachment of an analyte to the solid support.

As used herein, the term “flow cell” is intended to mean a vessel having a chamber where a reaction can be carried out, an inlet for delivering reagents to the chamber and an outlet for removing reagents from the chamber. In some embodiments, the chamber is configured for detection of the reaction that occurs in the chamber (e.g. on a surface that is in fluid contact with the chamber). For example, the chamber can include one or more transparent surfaces allowing optical detection of arrays, optically labeled molecules, or the like in the chamber. Exemplary flow cells include, but are not limited to those used in a nucleic acid sequencing apparatus such as flow cells for the Genome Analyzer®, MiSeq®, NextSeq® or HiSeq® platforms commercialized by Illumina, Inc. (San Diego, CA); or for the SOLID™ or Ion Torrent™ sequencing platform commercialized by Life Technologies (Carlsbad, CA). Exemplary flow cells and methods for their manufacture and use are also described, for example, in WO 2014/142841 A1; U.S. Pat. App. Pub. No. 2010/0111768 A1 and U.S. Pat. No. 8,951,781, each of which is incorporated herein by reference.

As used herein, the term “gel material” is intended to mean a semi-rigid material that is permeable to liquids and gases. Typically, a gel material can swell when liquid is taken up and can contract when liquid is removed, e.g., by drying. Exemplary gels include, but are not limited to, those having a colloidal structure, such as agarose; polymer mesh structure, such as gelatin; or cross-linked polymer structure, such as polyacrylamide, silane free acrylamide (see, for example, US Pat. App. Pub. No. 2011/0059865 A1), PAZAM (see, for example, U.S. Pat. No. 9,012,022, which is incorporated herein by reference), and polymers described in U.S. Patent Pub. Nos. 2015/0005447 and 2016/0122816, all of which are incorporated by reference in their entireties. Particularly useful gel material will conform to the shape of a well or other contours where it resides. Some useful gel materials can both (a) conform to the shape of the well or other contours where it resides and (b) have a volume that does not substantially exceed the volume of the well or contours where it resides. In some particular embodiments, the gel material is a polymeric hydrogel.

As used herein, the term “interstitial region” refers to an area in a substrate or on a surface that separates other areas of the substrate or surface. The interstitial region does not allow for the binding of library DNA. For example, an interstitial region can separate one library DNA binding region from another library DNA binding region. The two regions that are separated from each other can be discrete, lacking contact with each other. In some embodiments the interstitial region is continuous whereas the contours or features are discrete, for example, as is the case for an array of wells in an otherwise continuous surface. The separation provided by an interstitial region can be partial or full separation. Interstitial regions may have a surface material that differs from the surface material of the contours or features on the surface. For example, contours of an array can have an amount or concentration of gel material or analytes that exceeds the amount or concentration present at the interstitial regions. In some embodiments the gel material or analytes may not be present at the interstitial regions. In some embodiments, the DNA binding regions are inside an array of nanowells or capture pads, while the clustering primers (e.g., P5/P7 or P15/P17) are located on the interstitial regions separating the nanowells/capture pads. In other embodiments, the clustering primers are located inside a plurality or array of first nanowells, while the DNA binding regions are located inside a plurality or array of second nanowells that are smaller in size/dimension/diameter than the first nanowells, creating a well-within a well configuration. As such, the area within the first nanowell is considered to be an interstitial region of the second nanowell. In addition, the array of first nanowells are also separated by interstitial regions.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g., found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid, are intended as semantic identifiers for the nucleic acid in the context of a method or composition set forth herein and does not necessarily limit the structure or function of the nucleic acid beyond what is otherwise explicitly indicated. The terms “probe” and “target” can be similarly applied to other analytes such as proteins, small molecules, cells, or the like.

As used herein, the term “surface” is intended to mean an external part or external layer of a solid support or gel material. The surface can be in contact with another material such as a gas, liquid, gel, polymer, organic polymer, second surface of a similar or different material, metal, or coat. The surface, or regions thereof, can be substantially flat or planar. The surface can have surface contours such as wells, pits, channels, ridges, raised regions, pegs, posts or the like.

As used herein, the term “depression” refers to a discrete concave feature in a patterned support having a surface opening that is completely surrounded by interstitial region(s) of the patterned support surface. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. For example, the nanowell described herein are considered as a depression.

As used herein, the “solid support” or “substrate” may be used interchangeably and both refer to a rigid substrate that is insoluble in aqueous liquid. The substrate can be non-porous or porous. The solid support can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (e.g., acrylics, polystyrene and copolymers of styrene and other materials, polypropylene, polyethylene, polybutylene, polyurethanes, Teflon™, cyclic olefins, polyimides, etc.), nylon, ceramics, resins, Zeonor, silica or silica-based materials including silicon and modified silicon, carbon, metals, inorganic glasses, optical fiber bundles, and polymers. A particularly useful material is glass. Other suitable substrate materials may include polymeric materials, plastics, silicon, quartz (fused silica), boro float glass, silica, silica-based materials, carbon, metals including gold, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g. being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture. Further examples of materials that can be used in the structured substrates or methods of the present disclosure are described in US Pat. App. Pub. No. 2012/0316086 A1 and 2013/0116153, each of which is incorporated herein by reference.

As used herein, the term “well” refers to a discrete contour in a solid support having a surface opening that is completely surrounded by interstitial region(s) of the surface. Wells can have any of a variety of shapes at their opening in a surface including but not limited to round, elliptical, square, polygonal, star shaped (with any number of vertices), etc. The cross section of a well taken orthogonally with the surface can be curved, square, polygonal, hyperbolic, conical, angular, etc. In some embodiments, the well is a microwell or a nanowell.

As used herein, the term “clustering oligonucleotide” or “clustering primer” refers to nucleotide sequence immobilized on the surface of the solid support used for amplifying the template polynucleotides to create identical copies of the same templates (i.e., clusters). Examples of clustering oligonucleotide may include but not limited to P5 primer, P7 primer, P15 primer, P17 primer as described herein. In some embodiments, the “clustering primer” is also referred to as a “surface primer.”

The P5 and P7 primers are used on the surface of commercial flow cells sold by Illumina Inc. for sequencing on the Specific examples of suitable primers include P5 and/or P7 primers, which are used on the surface of commercial flow cells sold by Illumina, Inc., for sequencing on HISEQ™, HISEQX™, MISEQ™, MISEQDX™, MINISEQ™, NEXTSEQ™, NEXTSEQDX™, NOVASEQ™, GENOME ANALYZER™, ISEQ™, and other instrument platforms. These primers are also referred to as the clustering primers or clustering oligonucleotides. The primer sequences are described in U.S. Pat. Pub. No. 2011/0059865 A1, which is incorporated herein by reference. The P5 and P7 primer sequences comprise the following:

Paired end set:
P5: paired end 5′→3′
(SEQ ID NO. 1)
AATGATACGGCGACCACCGAGAUCTACAC
P7: paired end 5′→3′
(SEQ ID NO. 2)
CAAGCAGAAGACGGCATACGAGAT
Single read set:
P5: single read: 5′→3′
(SEQ ID NO. 3)
AATGATACGGCGACCACCGA
P7: single read 5′→3′
(SEQ ID NO. 4)
CAAGCAGAAGACGGCATACGA

In some embodiments, the P5 and P7 primers may comprise a linker or spacer at the 5′ end. Such linker or spacer may be included in order to permit cleavage, or to confer some other desirable property, for example to enable covalent attachment to a polymer or a solid support, or to act as spacers to position the site of cleavage an optimal distance from the solid support. In certain cases, 10-50 spacer nucleotides may be positioned between the point of attachment of the P5 or P7 primers to a polymer or a solid support. In some embodiments polyT spacers are used, although other nucleotides and combinations thereof can also be used. TET is a dye labeled oligonucleotide having complementary sequence to the P5/P7 primers. TET can be hybridized to the P5/P7 primers on a surface; the excess TET can be washed away, and the attached dye concentration can be measured by fluorescence detection using a scanning instrument such as a Typhoon Scanner (General Electric). In addition to the P5/P7 primers, other non-limiting examples of the sequencing primer sequences such as P15/P17 primers have also been disclosed in U.S. Publication No. 2019/0352327. These additional clustering primers comprise the following:

P15: 5′→3′
(SEQ ID NO. 5)
AATGATACGGCGACCACCGAGAT*CTACAC,
where T* refers to an allyl modified T
P17 primer 5′→3′
(SEQ ID NO. 6)
YYYCAAGCAGAAGACGGCATACGAGAT,
where Y is a diol linker subject to
chemical cleavage, for example, by
oxidation with a reagent such as
periodate, as disclosed in U.S.
Publication No. 2012/0309634, which
is incorporated by preference in its
entirety.

As used herein, the term “orthogonal” in the context of capturing library DNA complexes to surface, it is meant that the capture mechanism used to seed or attach the library DNA complex to the surface (e.g., capture moieties of the library DNA binding regions) of the solid support is different from the surface oligonucleotides (e.g., clustering primers in the interstitial regions) used for the clustering step.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

As used herein, “alkyl” refers to a straight or branched hydrocarbon chain that is fully saturated (i.e., contains no double or triple bonds). The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may be a larger size alkyl having 10 to 30 carbon atoms. The alkyl group may also be a medium size alkyl having 1 to 9 carbon atoms. The alkyl group could also be a lower alkyl having 1 to 6 carbon atoms. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkenyl” refers to a straight or branched hydrocarbon chain containing one or more double bonds. The alkenyl group may have 2 to 30 carbon atoms, although the present definition also covers the occurrence of the term “alkenyl” where no numerical range is designated. The alkenyl group may be a larger size alkenyl having 10 to 30 carbon atoms. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 30 carbon atoms, although the present definition also covers the occurrence of the term “alkynyl” where no numerical range is designated. The alkynyl group may be a larger size alkynyl having 10 to 30 carbon atoms. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “alkylene” means a branched, or straight chain fully saturated di-radical chemical group containing only carbon and hydrogen that is attached to the rest of the molecule via two points of attachment. The alkylene group may be a larger size alkylene having 10 to 30 carbon atoms. The alkylene group may also be a medium size alkylene having 1 to 9 carbon atoms. The alkylene group could also be a lower alkylene having 1 to 6 carbon atoms.

As used herein, the term “heteroalkylene” refers to an alkylene chain in which one or more skeletal atoms of the alkylene are selected from an atom other than carbon, e.g., oxygen, nitrogen, sulfur, phosphorus or combinations thereof.

As used herein, “alkenylene” means a straight or branched chain di-radical chemical group containing only carbon and hydrogen and containing at least one carbon-carbon double bond that is attached to the rest of the molecule via two points of attachment. The alkenylene group may be a larger size alkenylene having 10 to 30 carbon atoms. The alkenylene group may also be a medium size alkenylene having 2 to 9 carbon atoms. The alkenylene group could also be a lower alkenylene having 2 to 6 carbon atoms.

As used herein, “alkynylene” means a straight or branched chain di-radical chemical group containing only carbon and hydrogen and containing at least one carbon-carbon triple bond that is attached to the rest of the molecule via two points of attachment. The alkynylene group may be a larger size alkynylene having 10 to 30 carbon atoms. The alkynylene group may also be a medium size alkynylene having 2 to 9 carbon atoms. The alkynylene group could also be a lower alkynylene having 2 to 6 carbon atoms.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl. An aryl group may comprise one or more “substituents”, as described herein.

As used herein, “arylene” refers to an aromatic ring or ring system containing only carbon and hydrogen that is attached to the rest of the molecule via two points of attachment.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl. A heteroaryl group may comprise one or more “substituents”, as described herein.

As used herein, “heteroarylene” refers to an aromatic ring or ring system containing one or more heteroatoms in the ring backbone that is attached to the rest of the molecule via two points of attachment.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline. A heterocyclyl group may comprise one or more “substituents”, as described herein.

As used herein, “heterocyclylene” means a non-aromatic cyclic ring or ring system containing at least one heteroatom that is attached to the rest of the molecule via two points of attachment.

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “cycloalkylene” means a fully saturated carbocyclyl ring or ring system that is attached to the rest of the molecule via two points of attachment.

A “cycloalkenyl” or “cycloalkenylene” group refers to an alkenyl or alkenylene group respectively comprising a closed non-aromatic ring comprising from 3 to 10 carbon atoms, for example, 3 to 7 carbon atoms, and which contains at least one carbon-carbon double bond. A cycloalkenyl or cycloalkenylene group may comprise one or more “substituents”, as described herein.

A “cycloalkynyl” group refers to an alkynyl group respectively comprising a closed non-aromatic ring comprising from 3 to 12 carbon atoms, for example, 8 to 10 carbon atoms, and which contains at least one carbon-carbon triple bond. A cycloalkynyl group may comprise one or more “substituents”, as described herein.

As used herein, the term “azido” refers to a —N₃ group.

As used herein, the term “sulfur-based linkage” refers to a —(S)ₙ— group, wherein n is 1 to 10, or 1 to 6. Preferably, n can be 1, forming a “sulfide” linkage; or n is 2 to 10 (e.g. 2 to 6), forming a “polysulfide” linkage. For example, n is 2, forming a “disulfide” linkage.

In some embodiments, the sulfur atom may be optionally oxidised. In particular, a sulfur-based linkage may be a sulfone-S(═O)— linkage, or a sulfoxide —S(═O)₂-linkage.

As used herein, the term “acetal” refers to a —OC(R)(R′)O— group, where R and R′ are independently hydrogen or a “substituent” as described herein.

As used herein, the term “hemiaminal ether” refers to a —OC(R)(R′)NR″— group, where R, R′ and R″ are independently hydrogen or a “substituent” as described herein.

As used herein, the term “aminal” refers to a —NR(R′)(R″)NR′″— group, where R, R′, R″ and R′″ are independently hydrogen or a “substituent” as described herein.

As used herein, the term “imine” refers to a —C(R)═N— group, where R is hydrogen or a “substituent” as described herein.

As used herein, the term “hydrazone” refers to a —C(R)═N—NR′— group, where R and R′ are independently hydrogen or a “substituent” as described herein.

As used herein, the term “boron-based linkage” refers to a —(O)ₐ—B(OR)—(O)_{b}— group, where R is independently hydrogen or a “substituent” as described herein, and where a and b are independently 0 or 1.

As used herein, the term “silicon-based linkage” refers to a —(O)ₐ—Si(R)(R′)—(O)_{b}— group, where R and R′ are independently hydrogen or a “substituent” as described herein, and where a and b are independently 0 or 1.

As used herein, the term “phosphorus-based linkage” refers to a —(O)ₐ—P(R)—(O)_{b}— group, where R and R′ are independently hydrogen or a “substituent” as described herein, and where a and b are independently 0 or 1.

As used herein, the term “aldehyde” refers to a —C(═O) H group, where the group is attached to a carbon atom at the point of attachment to the group.

As used herein, the term “ketone” refers to a —C(═O)— group, where the group is attached to two other carbon atoms at the points of attachment to the group.

As used herein, the term “carboxylic acid” refers to a —C(═O) OH group. As used herein, the term “sulfonyl fluoride” refers to a —S(═O)₂F group.

As used herein, the term “diazo” refers to a —C(═N⁺═N⁻)— group.

As used herein, the term “oxime” refers to a —C(R)═N—OR′ group, where R and R′ are independently hydrogen or a “substituent” as described herein.

As used herein, the term “hydroximoyl halide” refers to a —C(X)═N—OR group, where R is a hydrogen or a “substituent” as described herein, and X is a halogen.

As used herein, the term “nitrile oxide” refers to a —C≡N⁺—O⁻ group.

As used herein the term “nitrone” refers to a —C(═NR⁺—O⁻)— group, where R is a hydrogen or a “substituent” as described herein.

When a group is described as “optionally substituted” it may be either unsubstituted or substituted. Likewise, when a group is described as being “substituted”, the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl)C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl)C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy)C₁-C₆ alkyl, —O(C₁-C₆ alkoxy)C₁-C₆ alkyl; (C₁-C₆ haloalkoxy)C₁-C₆ alkyl; —O(C₁-C₆ haloalkoxy)C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁₋₄alkyl, monophosphate, diphosphate, triphosphate, and oxo (═O). Wherever a group is described as “optionally substituted” that group can be substituted with the above substituents.

The embodiments set forth herein and recited in the claims can be understood in view of the above definitions.

### Methods of Seeding Library DNA

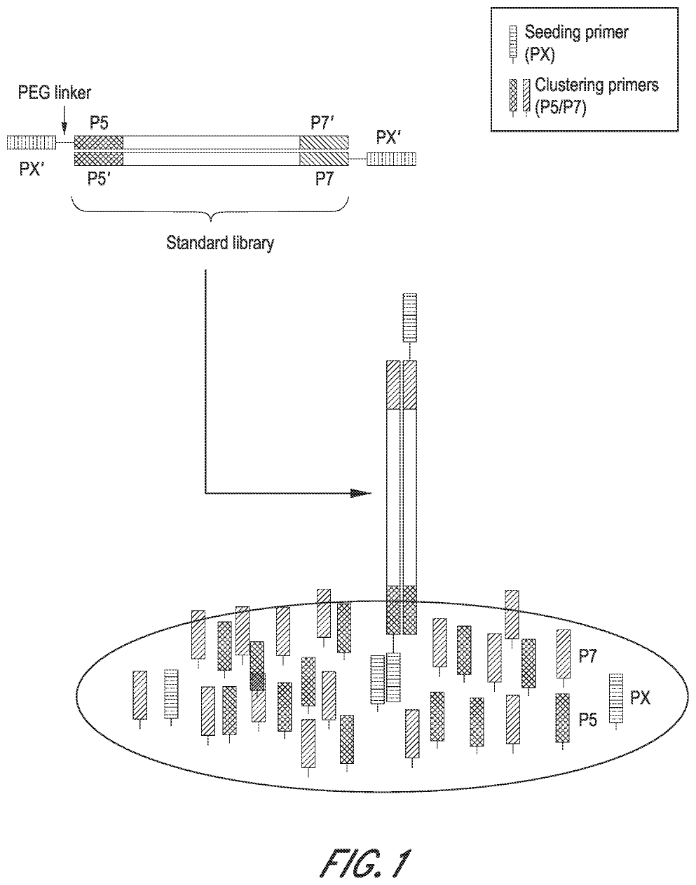

The library seeding methodology that is currently utilized on Illumina's SBS platforms relies on a hybridization event to capture library DNA on the flowcell surface. One embodiment of the seeding methodology is described in U.S. Ser. No. 63/128,663 and is illustrated in FIG. 1. In particular, this method relies on the use of a separate seeding primer that is orthogonal to the surface-bound clustering primers to attach the library DNA to a solid support. In FIG. 1, a double-stranded library DNA is furnished with adaptor sequences P5/P7′ and P5′/P7 and a separate seeding sequence PX′ via a PEG linker. A patterned solid support used for seeding the library DNAs comprises a plurality of nanowells, at least a portion of the nanowells each comprises a plurality of clustering primers (P5/P7 primers) and a number of capture primers each having the sequence PX (that is complementary to PX′). The PX′ bearing library DNA is then hybridized to the PX capture primer when a solution containing the library DNA is in contact with the solid support surface. The clustering primers on the solid support are used exclusively for amplifying the seeded library DNA samples.

The present disclosure provides an improved method to attach library DNAs to solid support. In particular, the method may result in no more than three library DNAs seeded in each distinctive DNA binding region (e.g., a nanowell or a capture pad) on the surface of the solid support. In some instance, the amplification of the library DNA on the solid support results in a monoclonal cluster or a single dominant cluster in at least 50%, 55%, 60%, 65%, 70%, 75%, 80%, 85%, 90%, 95%, 98%, or 99% of the library DNA binding regions. In some instance, at least 50%, 55%, 60%, 65%, 70%, 75%, 80%, 85%, 90%, 95%, 98%, or 99% of clusters generated from the amplification are monoclonal clusters.

Some aspect of the present disclosure relates a method of preparing a patterned surface of a solid support for sequencing, comprising:
contacting a solution comprising a plurality of library DNA complexes with the patterned surface comprising a plurality of library DNA binding regions separated by interstitial regions;
   wherein each library DNA binding region comprises a number of capture moieties adapted for capturing one library DNA complex, at least a portion of the interstitial regions comprise clustering oligonucleotides, and the capture moieties are orthogonal to the clustering oligonucleotides;
   wherein each library DNA complex comprises a single library polynucleotide and a number of accessory binding sites, and the number of accessory binding sites on each library DNA complex is more than the number of capture moieties in each library DNA binding region; and
attaching the plurality of library DNA complexes to the library DNA binding regions of the pattered surface by noncovalent or covalent interactions between the accessory binding sites of the library DNA complexes and the capture moieties of library DNA binding regions.

A library DNA (may also be referred to as template) as described herein may be of any suitable length, including for sequencing in an SBS process. For example, a library polynucleotide may be about 50 to 2000 nucleotides in length, about 75 to 1000 nucleotides in length, about 100 to 500 nucleotides in length, about 125 to 450 nucleotides in length, about 150 to 400 nucleotides in length, about 175 to 350 nucleotides in length, or about 200 to 300 nucleotides in length. In some embodiments, the library DNA complex comprises a single stranded library polynucleotide. In other embodiments, the library DNA complex comprises a double stranded library polynucleotide.

In some embodiments of the method described herein, the solid support comprises a flowcell. In some such embodiments, the flowcell is a patterned flowcell, and the flowcell surface containing a plurality of nanowells, at least a portion of the nanowells each comprises a single library DNA binding region that is inside the nanowell. In other embodiments, each of the library DNA binding region is a nanowell or a capture pad, forming a patterned surface of the flowcell. In further embodiments, the size, dimension, or diameter of each library binding regions is from about 10 nm to about 100 nm. In further embodiments, the size, dimension, or diameter of each library binding regions is about 10 nm, 20 nm, 30 nm, 40 nm, 50 nm, 60 nm, 70 nm, 80 nm, 90 nm, or 100 nm, or a range defined by any two of the preceding values. In further embodiments, the size, dimension, or diameter of each library binding regions is from about 20 nm to about 50 nm.

In some embodiments of the method described herein, each library DNA complex comprises a scaffold, wherein the number of accessory binding sites are on the scaffold, and the single library polynucleotide is attached to the scaffold by non-covalent or covalent interaction with a single template binding site on the scaffold.

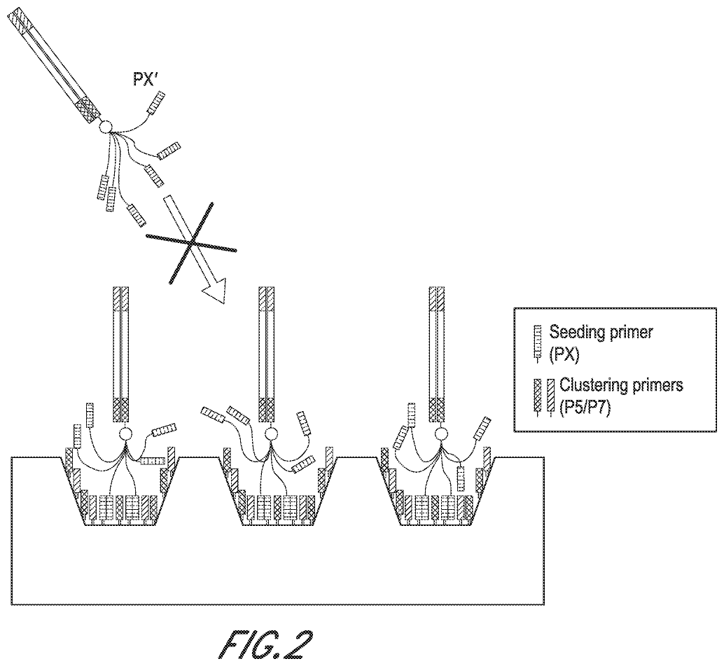

FIG. 2 illustrates an embodiment of the orthogonal seeding method described herein. First of all, a library DNA is furnished with a number of complementary seeding oligos (PX′) (i.e., accessory binding sites) attached to a standard adaptor sequence P5/P7 of a library DNA polynucleotide to form a library DNA complex. The number of PX′ moieties may be attached directly to the adaptor sequence via a linker (such as a PEG linker), or attached to the adaptor sequence via a scaffold. Alternatively, each of the accessory binding sites may be part of the scaffold. The patterned surface of a solid support has a plurality of nanowells. Within the nanowells, there are a number of surface-bound seeding primers (PX) that is complementary to PX′, and also a number of clustering primers (e.g., P5/P7). The number of accessory binding sites on each library DNA complex is more than the number of capture moieties within the DNA binding region of the nanowell. The library DNA complex is captured inside a nanowell by hybridizing the complementary seeding oligos (PX′) with the surface-bound seeding primers (PX). Once all the seeding primers PX within a nanowell are used by the PX′ binding sites of a library DNA complex, additional library DNA complexes are excluded from being hybridized to the same DNA binding region.

In some such embodiments of the method described herein, the scaffold of the DNA complex comprises or is a nanoparticle, a dendrimer, a polymer with bottlebrush structure, a single strand DNA scaffold, or a polypeptide scaffold, or a combination thereof. Nanoparticles comprising a scaffold, a single template site for bonding a template DNA to the scaffold, and a plurality of accessory sites for bonding accessory oligonucleotides to the scaffold have been described in U.S. Publication No. 2021/0187469 A1, which is incorporated by reference in its entirety. The scaffold may comprise one or a plurality of scaffold DNA molecules, such as a DNA dendrimer. In other embodiments, the scaffold may comprise a sing-stranded DNA. In still other embodiments, the scaffold may comprise one or more scaffold polypeptides. A polymer with bottlebrush structure may also be used as a scaffold, such as those described in U.S. Ser. No. 63/174,768. Other non-limiting example of a scaffold may comprise a single strand DNA produced by rolling circle amplification (RCA) that containing a plurality of accessory binding sites (e.g., an oligo sequence that is complementary to the seeding primer on the solid support).

### Scaffold

A scaffold may be synthesized so as to include, and may include once synthesized, more than one type of chemistry or structure for attachment. That is, it may be synthesized to include or be modified to include a single site of attachment to a library template polynucleotide, plus one or more additional bonding sites with a different chemistry or structure from the single library polynucleotide bonding site corresponding to accessory bonding sites.

In an example, a scaffold may be synthesized from one or more scaffold deoxyribonucleic acid (DNA) molecules. DNA molecules may be designed and structured as further disclosed herein so as to permit inclusion of different bonding sites (i.e., for a template polynucleotide as well as accessory binding sites) and also to provide size-exclusion properties for distancing template polynucleotides from each other once attached to a polynucleotide. In some examples, a scaffold may include a plurality of DNA molecules hybridized together so as to form a dendrimer. For example, adapters may be formed including a plurality of, such as three, strands of DNA, or oligodeoxyribonucleotide (oligo-DNA) molecules that can hybridize to each other by Watson-Crick base pairing so as to form a Y-shape, with one end of each hybridizing to one of the other two and the other end of each hybridizing to the other of the other two.

Such adapters may form a constitutional repeating until of a dendrimer. For example, each end of the Y-shaped adapter may have an overhang of DNA, where the end of one of the oligo-DNAs extends beyond the portion of which hybridizes to any other oligo-DNA. An adapter of one generation of such dendrimer may have an overhang on one end of the Y-shape, referred to here as the upstream end, that can hybridize with an overhang of an and of another Y adapter that constitutes a constitutional repeating unit of an immediately preceding generation of the dendrimer. And the other two ends of the adapter, referred to as the downstream ends, may each have an overhang that can hybridize with an overhang of an upstream end of a Y adapter that constitutes a constitutional repeating unit of an immediately following generation of the dendrimer. Thus, an adapter of one generation may attach to two adapters in the next generation, which may attach to four adapters of the following generation, which may attach to eight adapters of the following generation, and so on. Any one end of one of the terminal Y adapters, whether a downstream overhang of any generation, such as the last generation, not hybridized to an upstream overhang of another adapter, or the upstream overhang of the first generation, may include or be attached to the single template polynucleotide binding site. In an example, the DNA-oligo including the upstream overhang of the first generation adapted may itself be an extension of a template polynucleotide, added thereto during sample preparation. Other ends or overhangs may include or be attached to accessory sites.

In some examples, a scaffold may include one or more single-stranded DNA (ssDNA) molecules modified or structured so as to permit attachment thereto of a single library polynucleotide and one or more accessory compositions or a structure, to one or more accessory bonding site. Various methods for producing an ssDNA-based scaffold may be used. In an example, a double-stranded closed loop or plasmid may serve as a coding sequence for an ssDNA scaffold molecule, in a rolling circle amplification process. Replication of a strand thereof by a strand-displacing DNA polymerase (e.g., Phi29) may produce an ssDNA molecule including concatemerized copies of the copied strand of the circular coding strand. Reaction conditions may be adopted so as to result in synthesis of an ssDNA scaffold of a desired size. A 5-prime or 3-prime end may be further modified to include or be attached or attachable to a single library template polynucleotide molecule, as the single template site. Accessory binding sites may include the other end of the ssDNA scaffold molecule, or modifications to or of individual nucleotides of the strand.

In another example, an ssDNA scaffold may be synthesized by use of a template-independent polymerase (e.g., terminal deoxynucleotidyl transferase, or TdT). TdT incorporates deoxynucleotides at the 3-prime-hydroxyl terminus of a single-stranded DNA strand, without requiring or copying a template. A 5-prime or 3-prime end may be further modified to include or be attached or attachable to a single library polynucleotide molecule, as the single template site. Accessory sites may include the other end of the ssDNA scaffold molecule, or modifications to or of individual nucleotides of the strand.

In another example, an ssDNA scaffold may be synthesized by producing a plurality of single-stranded DNA molecules by any applicable method and ligating them together to form a single ssDNA molecule as a scaffold. For example, a polymerase may polymerize formation of a nascent strand of DNA by copying a linearized DNA coding strand, in a run-off polymerization reaction (i.e., where the polymerase ceases extending a nascent strand upon reaching a 5-prime end of a coding strand). A plurality of ssDNA products may be synthesized, then ligated end-to-end for formation of a single ssDNA scaffold. In an example, ligation of one ssDNA product to another may be accomplished with the aid of a splint. For example, a short oligo-DNA may be designed whose 3-prime end is complementary of the 5-prime end of one ssDNA product and whose 5-prime end is complementary to the 3-prime end of another ssDNA product, such that hybridization of the DNA-oligo to the two ssDNA products brings the 5-prime end of one together with the 3-prime end of the other in a nicked, double-stranded structure where they meet hybridized to the DNA-oligo. A DNA ligase (e.g., T4) may then be used to enzymatically ligate the two ends together to form a single ssDNA molecule from the two. Additional reactions may be included with DNA-oligos for splint-aided ligation of one or both ends of the product of such first reaction to another ssDNA product, and so on, for construction of an ssDNA scaffold as may be desired.

In some embodiments of the method described herein, the library polynucleotide is attached to the scaffold by noncovalent interaction, such as hybridization with the single template binding site. In other embodiments, the noncovalent interaction between the library polynucleotide and the scaffold may be avidin-biotin interaction. For example, each of the library polynucleotides comprises a biotin moiety that allows for non-covalent bonding with streptavidin binding site located on the scaffold. In other embodiments, biotin moiety on the library polynucleotide and the streptavidin binding site on the scaffold may be reversed.

In some other embodiments of the method described herein, the library polynucleotide is attached to the scaffold by covalent bonding with the single template binding site on the scaffold. In some such embodiments, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

In further embodiments, the template binding site on the scaffold may comprise amino bonding sites, carboxy bonding sites, thiol bonding sites, aldehyde bonding sites, azido bonding sites, hydroxy bonding sites, cycloalkene bonding sites (such as transcyclooctene bonding sites or norbornene bonding sites), cycloalkyne bonding sites (such as cyclooctyne bonding sites dibenzocyclooctyne (DBCO) bonding sites, or bicyclononyne bonding sites), oxoamine bonding sites, SpyTag bonding sites, Snap-tag bonding sites, CLIP-tag bonding sites, or proteins with N-terminus recognized by sortase, or combinations thereof. In some such embodiments, each of the library polynucleotides or template polynucleotides comprises a functional moiety that allows for covalent bonding with the template bonding site of the scaffold. The functional moiety of the library polynucleotides may comprise or is selected from a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other embodiments, the functional moiety of the template bonding site on the scaffold and the functional moiety of the library polynucleotide may be reversed.

A non-exclusive list of complementary binding partners is presented in Table 1:

TABLE 1

| | Exemplary template bonding site | Exemplary functional moiety on the |
|---|---|---|
| | on the scaffold or functional | library polynucleotide or template |
| Bonding site | moiety on library polynucleotide | bonding site on the scaffold |
| amine-NHS | amine group, —NH₂ | N-Hydroxysuccinimide ester |
| | | 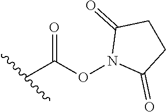 [SMILES: *C(=O)ON1C(=O)CCC1=O] |
| amine- | amine group, —NH₂ | imidoester |
| imidoester | | 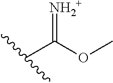 [SMILES: *C(=[NH2+])OC] |
| amine- | amine group, —NH₂ | pentofluorophenyl ester, |
| pentofluoro- phenyl ester | | 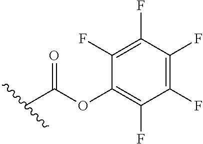 [SMILES: *C(=O)Oc1c(F)c(F)c(F)c(F)c1F] |
| amine- | amine group, —NH₂ | hydroxymethyl phosphine |
| hydroxymethyl phosphine | | 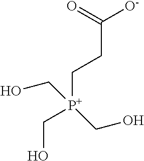 [SMILES: O=C([O-])CC[P+](CO)(CO)CO] |
| amine- | amine group, —NH₂ | carboxylic acid group, —C(═O)OH (e.g., |
| carboxylic | | following activation of the carboxylic |
| acid | | acid by a carbodiimide such as EDC (1- |
| | | ethyl-3-(-3-dimethylaminopropyl) |
| | | carbodiimide hydrochloride) or DCC |
| | | (N′,N′-dicyclohexyl carbodiimide) to |
| | | allow for formation of an amide bond |
| | | of the activated carboxylic acid with an |
| | | amine group) |
| thiol- | thiol, —SH | maleimide |
| maleimide | | 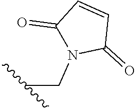 [SMILES: *CN1C(=O)C=CC1=O] |
| thiol- | thiol, —SH | haloacetyl (e.g., iodoacetyl or other |
| haloacetyl | | haloacetyl) |
| | | 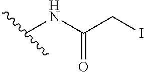 [SMILES: *NC(=O)CI] |
| thiol-pyridyl | thiol, —SH | pyridyl disulfide |
| disulfide | | 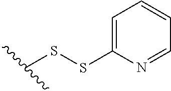 [SMILES: *SSc1ccccn1] |
| thiol- | thiol, —SH | thiosulfonate |
| thiosulfonate | | 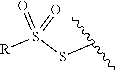 |
| thiol-vinyl | thiol, —SH | vinyl sulfone |
| sulfone | | 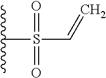 [SMILES: *S(=O)(=O)C=C] |
| aldehyde- | aldehyde, —C(═O)H | hydrazide |
| hydrazide | | 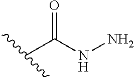 [SMILES: *C(=O)NN] |
| aldehyde- | aldehyde, —C(═O)H | alkoxyamine |
| alkoxyamine | | 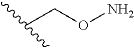 [SMILES: *CON] |
| hydroxy- | hydroxyl, —OH | isocyanate |
| isocyanate | | 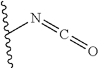 [SMILES: *N=C=O] |
| azide-alkyne | azide, —N₃ | alkyne |
| | | 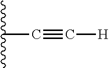 [SMILES: *C#C] |
| azide- | azide, —N₃ | phosphine, e.g.: |
| phosphine | | 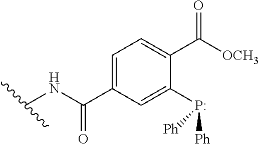 |
| azide- | azide, —N₃ | cyclooctyne, e.g. dibenzocyclooctyne |
| cyclooctyne | | (DBCO) |
| | | 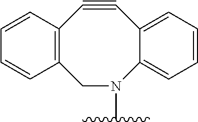 [SMILES: *N1Cc2ccccc2C#Cc2ccccc21] |
| | | or BCN (bicyclo[6.1.0]nonyne) |
| | | 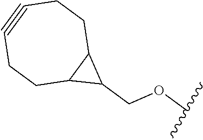 [SMILES: *OCC1C2CCC#CCCC21] |
| azide- | azine, —N₃ | norbornene |
| norbornene | | 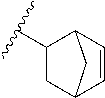 [SMILES: *C1CC2C=CC1C2] |
| transcyclo- | transcyclooctene | tetrazine, e.g., benzyl-methyltetrazine |
| octene-tetrazine | 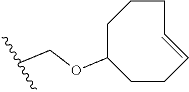 [SMILES: *COC1CC/C=C/CCC1] | 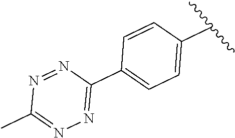 [SMILES: *c1ccc(-c2nnc(C)nn2)cc1] |
| norbornene- | norbornene | tetrazine, e.g. benzyl-tetrazine |
| tetrazine | 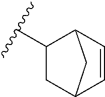 [SMILES: *C1CC2C=CC1C2] | 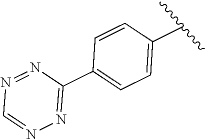 [SMILES: *c1ccc(-c2nncnn2)cc1] |
| oxime | aldehyde or ketone (e.g., amine | alkoxyamine |
| | group or N-terminus of polypeptide | |
| | converted to an aldehyde or ketone | |
| | by pyroxidal phosphate) | |
| SpyTag- | SpyTag: amino acid sequence | SpyCatcher amino acid sequence: |
| SpyCatcher | AHIVMVDAYKPTK | MKGSSHHHHHHVDIPTTENLYFQ |
| | | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK |
| SNAP-tag-O⁶- | SNAP-tag (O-6-methylguanine- | O⁶-Benzylguanine |
| Benzylguanine | DNA methyltransferase) | 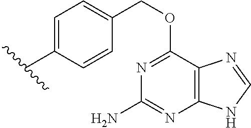 [SMILES: *c1ccc(COc2nc(N)nc3[nH]cnc23)cc1] |
| CLIP-tag-O²- | CLIP-tag (modified O-6- | O²-benzylcytosine |
| benzylcytosine | methylguanine-DNA methyltransferase) | 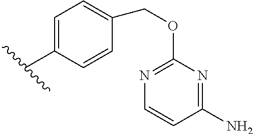 [SMILES: *c1ccc(COc2nccc(N)n2)cc1] |
| Sortase- | -Leu-Pro-X-Thr-Gly | -Gly₍₃₋₅₎ |
| coupling | | |

In some embodiments of the method described herein, each library DNA complex is attached to the DNA binding region of the solid support by non-covalent interaction between the capture moieties and the accessory binding sites of the library DNA complex. For example, each capture moiety of the DNA binding region comprises an oligonucleotide seeding sequence (also referred to as a seeding primer, e.g., PX), and the accessory binding sites of the library DNA complex each comprises an oligo sequence that is complementary or at least partially complementary to the seeding primer (e.g., PX′). The library DNA complex is attached to the library DNA binding region by hybridization of one or more accessory binding sites of the library DNA complex with the oligonucleotide seeding sequences (seeding primers). In some such embodiments, each seeding primers comprises from about 10 to about 40 nucleotides, or from about 20 to about 30 nucleotides. In other embodiments, each capture moiety comprises an avidin moiety, the accessory binding sites on the library DNA complex each comprises a biotin moiety, and the library DNA complex is attached to the library DNA binding region by avidin (e.g. streptavidin) biotin interaction. In other embodiments, the avidin biotin interactions may be reversed.

In some other embodiments of the method described herein, each library DNA complex is attached to the library DNA binding region by covalent bonding. The covalent bonding may include but not limited to amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding. In further embodiments, the capture moieties may comprise amino bonding sites, carboxy bonding sites, thiol bonding sites, aldehyde bonding sites, azido bonding sites, hydroxy bonding sites, cycloalkene bonding sites (such as transcyclooctene bonding sites or norbornene bonding sites), cycloalkyne bonding sites (such as cyclooctyne bonding sites dibenzocyclooctyne (DBCO) bonding sites, or bicyclononyne bonding sites), oxoamine bonding sites, SpyTag bonding sites, Snap-tag bonding sites, CLIP-tag bonding sites, or proteins with N-terminus recognized by sortase, or combinations thereof. In some such embodiments, each of the accessory binding sites on the library DNA complex comprises a functional moiety that allows for covalent bonding with capture moieties of the DNA binding region. The functional moiety of the accessory binding sites of the library DNA complex may comprise or is selected from a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In one example, the capture moieties of the solid support comprises azido groups and the accessory binding sites of the library DNA complex comprise dibenzocyclooctyne (DBCO) moiety, which undergoes strain-promoted copper-free click reaction to form covalent bonding. Additional exemplary embodiments of the complementary partners (between the capture moieties and the accessory binding sites of the library DNA complex) are presented in Table 1 above. In other embodiments, the functional moiety of capture moieties on the solid support and the functional moiety of the accessory binding sites of the library DNA complex may be reversed.

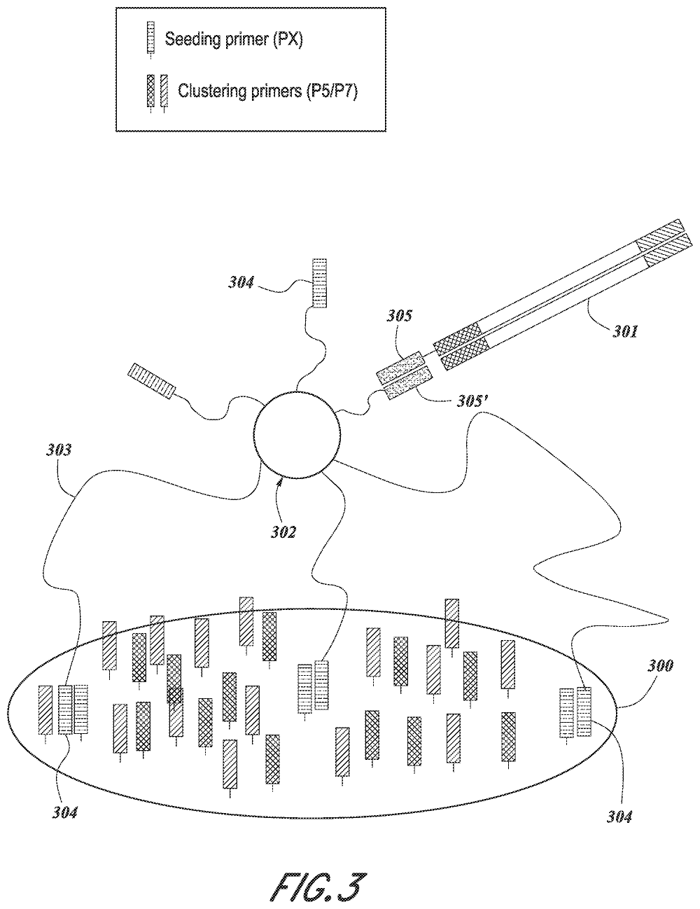

FIG. 3 is an amplified view of a library DNA complex described herein attached to a nanowell 300 of a solid support. A library DNA 301 comprises a double-stranded library polynucleotide, adaptor sequences, and a scaffold 302. A number of accessory binding sites PX′ oligos 304 are attached to scaffold 302. Each oligo 304 may be attached to scaffold 302 through an optional linker 303. Alternatively, each oligo 304 and linker 303 is a part of the structure of scaffold 302. For example, scaffold 302 may be a dendrimer, a polymer with bottlebrush structure, a single strand DNA scaffold, or a polypeptide scaffold. At least a portion of the accessory binding sites 304 are each hybridized to the surface bound seeding primers inside the nanowell 300 such that all available seeding primers inside the nanowell 300 are used or occupied (through hybridization with PX′).

In some embodiments of the method described herein, each library DNA binding region may comprise from about 1 to 200 capture moieties. In some further embodiments, each library DNA binding region may comprise about 10, 20, 30, 40, 50, 60, 70, 80, 90, 100, 110, 120, 130, 140, 150, 160, 170, 180, 190 or 200 moieties, or a range defined by any of the two preceding values. In some embodiments, the ratio of the clustering oligonucleotides to the capture moieties is from about 10 to 100,000. In further embodiments, the ratio of the clustering oligonucleotides to the capture moieties is about 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000, or 10,000, or a range defined by any two of the preceding values.

In some embodiments of the method described herein, the clustering oligonucleotides comprise P5 and P7 primers, or P15 and P17 primers.

A standard library seeding method on the patterned flowcell with nanowells generally results in about 2 to 5 library DNAs (template polynucleotides) seeded per nanowell, in at least a portion of the nanowells. Using the method described in the present application, it may improve the seeding to no more than 3 library DNA complexes per DNA binding region (e.g., a nanowell or a capture pad), which may improve monoclonality. In further embodiments, wherein at least 50%, 55%, 60%, 65%, 70%, 75%, 80%, 85%, 90%, 95%, 98%, or 99% of the library DNA binding regions are each occupied with one or two library DNA complexes. In still further embodiments, wherein at least 50%, 55%, 60%, 65%, 70%, 75%, 80%, 85%, 90%, 95%, 98%, or 99% of the library DNA binding regions are each occupied with a single library DNA complex or only a single dominant library DNA complex.

In some embodiments, the method further comprises amplifying the library polynucleotides. In some embodiments, amplification of the library DNA complex in at least 50%, 55%, 60%, 65%, 70%, 75%, 80%, 85%, 90%, 95%, 98%, or 99% of the DNA binding regions results in a monoclonal cluster or a single dominant cluster. In some embodiments, at least 50%, 55%, 60%, 65%, 70%, 75%, 80%, 85%, 90%, 95%, 98%, or 99% of clusters generated from the amplification are monoclonal clusters.

### Library DNA Preparation

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adaptors (adaptor sequences). This may be followed by amplification and sequencing. The original sample DNA fragments are referred to as “inserts.” Alternatively, “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size fractionated prior to modification with the adaptor sequences.

As used herein, an “adaptor” sequence comprises a short sequence-specific oligonucleotide that is ligated to the 5′ and 3′ ends of each DNA (or RNA) fragment in a sequencing library as part of library preparation.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridized to a complementary strand—for example, a long polynucleotide strand hybridized to a short nucleotide primer—it may still be referred to herein as a single stranded nucleic acid.

Some embodiments of the present disclosure relate to a DNA library comprising a plurality of library DNA complexes, each library DNA complex comprises a single library polynucleotide, and a scaffold comprising a number of accessory binding sites adapted for attaching to a number of capture moieties on a library DNA binding region of a patterned solid support, wherein the library polynucleotide comprises an insert and an adaptor region;

wherein the adaptor region comprises a clustering sequence, each accessory binding sites comprises a complementary capture moiety, wherein the clustering sequence and the complementary capture moiety are orthogonal; and wherein the number of accessory binding sites on each library DNA complex is more than the number of capture moieties in the library DNA binding region.

In some embodiments of the DNA library described herein, the library DNA complex further comprises a spacer region between the clustering sequence and the scaffold. In some such embodiments, the spacer region comprises a linker. In further embodiments, the linker comprises a PEG linker.

In some embodiments of the DNA library described herein, the library polynucleotide further comprises an index sequence (e.g., i5), a first sequencing binding site (e.g., SBS3), a second sequencing binding site (e.g., SBS12), and/or a second index sequence (e.g., i7).

In some embodiments of the DNA library described herein, the adaptor region comprises a P5, P7, P15 or P17 sequence, or a sequence that is complementary, or at least partially complementary to P5, P7, P15 or P17 sequence (i.e., P5′, P7′, P15′ or P17′ sequence). In some embodiments, the P5/P7 or P15/P17 sequence (or complementary sequence thereof) of the library DNA complex described herein, may be attached to a scaffold comprising a number of accessory binding sites, each accessory binding site comprises an oligo sequence (e.g., PX′) that is orthogonal to the sequence in the adaptor region. Such accessory binding site oligo sequence is used for attaching the DNA complex to the capture moieties on the solid support. The corresponding capture moieties (e.g., a seeding primer or a seeding oligonucleotide) (e.g., PX) on the solid support each comprises a sequence that is complementary or at least partially complementary to the oligo sequence of accessory binding site. Non-limiting examples of the seeding primer sequence and the complementary sequence of the accessory binding sites may include those disclosed in U.S. Ser. No. 63/128,663, such as the following:

PX′:
(SEQ ID NO. 7)
CCTCCTCCTCCTCCTCCTCCTCCT
PX
(SEQ ID NO: 8)
AGGAGGAGGAGGAGGAGGAGGAGG
PA
(SEQ ID NO: 9)
GCTGGCACGTCCGAACGCTTCGTTAATCCGTTGAG
cPA (PA′)
(SEQ ID NO: 10)
CTCAACGGATTAACGAAGCGTTCGGACGTGCCAGC
PB
(SEQ ID NO: 11)
CGTCGTCTGCCATGGCGCTTCGGTGGATATGAACT
cPB (PB′)
(SEQ ID NO: 12)
AGTTCATATCCACCGAAGCGCCATGGCAGACGACG
PC
(SEQ ID NO: 13)
ACGGCCGCTAATATCAACGCGTCGAATCCGCAACT
cPC (PC′)
(SEQ ID NO: 14)
AGTTGCGGATTCGACGCGTTGATATTAGCGGCCGT
PD
(SEQ ID NO: 15)
GCCGCGTTACGTTAGCCGGACTATTCGATGCAGC
cPD (PD′)
(SEQ ID NO: 16)
GCTGCATCGAATAGTCCGGCTAACGTAACGCGGC

In some embodiments of the DNA library described herein, the DNA library is a double-stranded library. In other embodiments, the DNA library is a single-stranded library.

In some embodiments of the DNA library described herein, the scaffold comprises a DNA dendrimer, a polymer with bottlebrush structure, a single strand DNA with bottlebrush structure, a single strand DNA produced by rolling circle amplification, or a polypeptide scaffold, or a combination thereof. In some further embodiments, each complementary capture moiety of the library DNA complex comprises an oligo sequence that is hybridizable to the capture moiety on the library binding region of the patterned solid support. For example, each capture moieties on the solid support comprises PX seeding primer sequence, and each complementary capture moiety of the library DNA complex comprises PX′ sequence that is complementary to PX, which allows for hybridization between the capture moieties and the accessory binding sites.

In some embodiments of the DNA library described herein, library DNA strands are modified to possess a number of accessory binding sites that are capable of being captured at the surface of the solid support, for example, being captured by the capture moieties of the DNA binding regions described herein. There are two exemplary workflows for preparing modified DNA library: (A) PCR-based libraries and (B) PCR-free libraries. In some embodiments, the chemical functionalization of the library DNA can be incorporated covalently through a round of PCR amplification prior to clustering (PCR-based libraries) or added through modification to existing workflow steps including adapter hybridization during library preparation (PCR-free libraries).

In addition to the sequences that are complementary to the clustering primers (such as P5′/P7′, or P15′/P17′), additional sequences may be added to the library strands. The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analyzed in a single run, without drastically increasing run cost or run time. Examples of tag sequences are found in WO 2005/068656, whose contents are incorporated herein by reference in their entirety. The tag can be read at the end of the first read by hybridizing an index read primer, or at the end of the second read, by using the surface primers as index read primers P7. The invention is not limited by the number of reads per cluster, for example two reads per cluster: three or more reads per cluster are obtainable simply by rehybridizing a first extended sequencing primer, and rehybridizing a second primer before or after a cluster repopulation/strand resynthesis step. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e., hybridizes) to a portion of the sequencing binding site on the template strand. The DNA polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g., SBS3′) followed by synthesis and sequencing of the complementary strand. This leads to the sequencing of the insert. In a second step, an index sequencing primer (e.g., i7 sequencing primer) binds to a second sequencing binding site (e.g., SBS12) leading to synthesis and sequencing of the index sequence (e.g., sequencing of the i7 primer). The second sequencing read may comprise binding of an index sequencing primer (e.g., i5 sequencing primer) to the complement of the first sequencing binding site on the template (e.g., SBS3) and synthesis and sequencing of the index sequence (e.g., i5). In a second step, a second sequencing primer (read 2 sequencing primer) binds to the complement of the primer (e.g., i7 sequencing primer) binds to a second sequencing binding site (e.g., SBS12′) leading to synthesis and sequencing of the insert in the reverse direction.

Once a double stranded nucleic acid template library is formed, typically, the library will be subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.). In one embodiment, chemical denaturation, such as NaOH or formamide, is used. In another embodiment, the DNA is thermally denatured by heating.

Following denaturation, a single-stranded template library can be contacted in free solution onto a solid support comprising surface capture moieties (for example P5 and P7 primers). This solid support is typically a flowcell, although in alternative embodiments, seeding and clustering can be conducted off-flowcell using alternative solid support.

In addition to the capture mechanism described in FIG. 3, alternative embodiments immobilizing library DNA complex to the DNA binding regions of the solid support are described herein. In one embodiment, a single or double stranded library DNA complex may comprise a plurality of biotin moieties (i.e., accessory binding sites) that can form noncovalent bonding with avidin moieties on the surface of the solid support. Alternatively, the solid support may comprise biotin capture moieties and the library DNA may be functionalized with a plurality of avidin moieties (e.g., streptavidin). Other non-covalent interaction may also be used. These non-covalent interactions may include one or more of ionic bonds, hydrogen bonds, hydrophobic interactions, π-π interactions, van der Waals interactions and host-guest interactions described herein. Where non-covalent interactions are used, the type of interaction is not particularly limited, provided that the interactions are (collectively) sufficiently strong for the template to remain attached to the solid support during extension. The non-covalent interactions may also be weak enough such that the template can then be removed from the solid support once a copy of the template has been extended on a surface primer.

Numerous methods are available for including one or more biotin moiety in or adding one or more biotin moieties to a library DNA. For example, biotinylated nucleotides are commercially available for incorporation into a DNA molecule by a polymerase, and kits are commercially available for adding a biotin moiety to a polynucleotide or a polypeptide. Biotin residues can also be added to amino acids or modified amino acids or nucleotides or modified nucleotides. Linking chemistries shown in Table 1 can also be used for adding a biotin group to proteins such as on carboxylic acid groups, amine groups, or thiol groups. Several biotin ligase enzymes are also available for enzymatically targeted biotinylation such as of polypeptides (e.g., of the lysine reside of the AviTag amino acid sequence GLNDIFEAQKIEWHE included in a polypeptide). A genetically engineered ascorbate peroxidase (APEX) is also available for modifying biotin to permit biotinylation of electron-rich amino acids such as tyrosine, and possibly tryptophan, cysteine, or histidine.

In another example, a polypeptide including the amino acid sequence DSLEFIASKLA may be biotinylated (at the more N-terminal of the two S residues present in the sequence), which is a substrate for Sfp phosphopantetheinyl transferase-catalyzed covalent attachment thereto with small molecules conjugated to coenzyme A (CoA). For example, a polypeptide including this sequence could be biotinylated through covalent attachment thereto by a CoA-biotin conjugate. This system may also be used for attaching many other types of bonding moieties or structures identified in Table 1 for use in creating bonding sites for a scaffold to bond to a DNA molecule or polypeptide or other molecule as disclosed herein. For example, a CoA conjugated to any of the reactive pair moieties identified in Table 1 could be covalently attached to a polypeptide containing the above-identified sequence by Sfp phosphopantetheinyl transferase, thereby permitting bonding of another composition thereto that includes the complementary bonding partner.

Other enzymes may be used for adding bonding moiety to a polypeptide. For example, a lipoic acid ligase enzyme can add a lipoic acid molecule, or a modified lipoic acid molecule including a bonding moiety identified in Table 1 such as an alkyne or azide group, can be covalently linked to the amine of a side group of a lysine reside in an amino acid sequence DEVLVEIETDKAVLEVPGGEEE or GFEIDKVWYDLDA included in a polypeptide. In another example, a scaffold, template polynucleotide, or other polypeptide or DNA molecule included therein or intended to be bonded thereto may include or be attached to an active serine hydrolase enzyme. Fluorophosphonate molecules become covalently linked to serine residues in the active site of serine hydrolase enzymes. Commercially available analogs of fluorophosphonate molecules including bonding moieties identified in Table 1, such as an azide group or a desthiobiotin group (an analog of biotin that can bind to avidin). Thus, such groups can be covalently attached to serine hydrolase enzyme included in or attached to a polypeptide or DNA molecule used in or attached to a scaffold as disclosed herein and such bonding moiety or structure can be covalently added thereto by use by attachment of a suitable modified fluorophosphonate molecule for creating a bonding site on such protein for a complementary bonding partner from Table 1 (such as for azide-alkyne, azide-phosphine, azide-cyclooctyne, azide-norbornene, or desthiobiotin-avidin bonding).

In another embodiment, the library DNA complex may be attachable to the solid support by covalent bonds. The surface of the solid support comprises a plurality of azido moieties (e.g., the azido groups of a PAZAM coated surface). The single or double stranded library DNA complex is modified to include a plurality of DBCO functionality

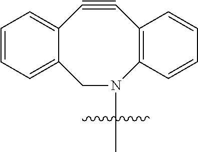 [SMILES: *N1Cc2ccccc2C#Cc2ccccc21]

as accessory binding sites. The library DNA is covalently bounded to the surface by reaction of the DBCO and azido groups, forming

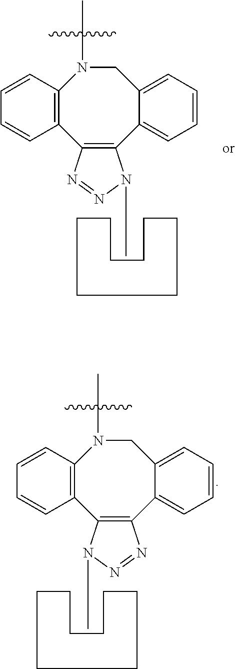

Where covalent bonds are used, the bond may be stable such that the template remains attached to the solid support. Non-limiting examples of covalent bonds include alkylene linkages, alkenylene linkages, alkynylene linkages, ether linkages (e.g. ethylene glycol, propylene glycol, polyethylene glycol), amine linkages, ester linkages, amide linkages, carbocyclic or heterocyclic linkages, sulfur-based linkages (e.g. thioether, disulfide, polysulfide, or sulfoxide linkages), acetals, hemiaminal ethers, aminals, imines, hydrazones, boron-based linkages (e.g. boronic and borinic acids/esters), silicon-based linkages (e.g. silyl ether, siloxane), and phosphorus-based linkages (e.g. phosphite, phosphate).

In some embodiments, the covalent bond may be a reversible covalent bond such that the template can then be removed from the solid support once a copy of the library DNA has been extended on a surface primer. In other embodiments, the covalent bond may be a non-reversible bond.

Any suitable bioconjugation methods for adding functional moiety to the library polynucleotides or surface primers may be used. Modified nucleotides may be commercially available possessing the functional moieties or structures, and methods for attaching or including them to polymer, a nucleotide, or polynucleotide are also known. Bifunctional linker molecules with a moiety or structure from one complementary pair of bonding partners listed in Table 1 at one end and a moiety or structure from another complementary pair of bonding partners listed in Table 1 may also be commercially available. The library polynucleotide or the clustering oligonucleotides may be bound to one end of such a linker, resulting in the initial moiety or structure being effectively replaced with another, i.e., the moiety or structure present on the other end of the linker.

For example, a bifunctional linker may have on one end a moiety from among those listed in Table 1, such as an NHS-ester group. At the other end it may have another group, such as an azido group. The ends may be connected to each other by a linker, such as, for example, one or more PEG groups, alkyl chain, combinations thereof in a linking sequence, etc. If a library polynucleotide has an amine group, the NHS-ester end of the bifunctional linker can be bound to the amine group, leaving the free azido end available for bonding to the first plurality of bonding sites bearing a bonding partner for an azido group (e.g., alkyne, phosphine, cyclooctyne, or norbornene, etc.). Many other examples of bifunctional linkers are commercially available including on an end a moiety identified in Table 1 for forming one type of bonding site or functional moiety and on the other end a different moiety identified in Table 1 for forming another type of bonding site or functional moiety.

In another example, the library DNA complex may include a plurality of first polypeptide sequence as accessory binding sites, and the capture moieties of the solid support may have a second polypeptide sequence capable of covalently bonding to the first polypeptide sequences of the library DNA complex. Non-limiting examples of such pairs include the SpyTag/SpyCatcher system, the Snap-tag/O⁶-Benzylguanine system, and the CLIP-tag/O²-benzylcytosine system. Similarly, the surface primer oligonucleotides and the second plurality of the bonding sites of the substrate may have the first polynucleotide sequence and the second polynucleotide sequence. Amino acid sequences for the complementary pairs of the SpyTag/SpyCatcher system and polynucleotides encoding them may be available. Examples of sequences are provided in Table 1. Several amino acid site mutations for a SpyTag sequence and for a SpyCatcher sequence may be available for inclusion in recombinant polypeptides. A Snap-tag is a functional O⁶-methylguanine-DNA methyltransferase, and a CLIP-tag is a modified version of Snap-tag. Nucleotide sequences encoding Snap-tag, CLIP-tag, SpyCatcher, may be commercially available for subcloning and inclusion in engineered polypeptide sequences.

Alternatively, the library DNA complex may be covalently attached to the capture moieties of the solid support each other via an enzymatically catalyzed formation of a covalent bond. For example, a library DNA complex and the capture moieties may include motifs capable of covalent attachment to each other by sortase-mediated coupling, e.g. a LPXTG amino acid sequence on one and an oligo glycine nucleophilic sequence on the other (with a repeat of, e.g., from 3 to 5 glycines). Sortase-mediated transpeptidation may then be carried out to result in covalent attachment of the library DNA complex at the DNA binding region.

In another example, the library DNA complex may comprise peptide sequences that may bind to peptide sequences (capture moieties) on the solid support as complementary pairs of a coiled coil motif. A coiled coil motif is a structural feature of some polypeptides where two or more polypeptide strands each form an alpha-helix secondary structure and the alpha-helices coil together to form a tight non-covalent bond. A coiled coil sequence may include a heptad repeat, a repeating pattern of the seven amino acids HPPHCPC (where H indicates a hydrophobic amino acid, C typically represents a charged amino acid and P represents a polar, hydrophilic amino acid). An example of a heptad repeat is found in a leucine zipper coiled coil, in which the fourth amino acid of the heptad is frequently leucine.

Any of the foregoing methods of biotinylating compositions to promote bonding to a polypeptide including an avidin sequence (such as an avidin polypeptide included in or attached to another composition), or otherwise adding functional groups to polypeptides, as part of a scaffold, attached to a scaffold, part of an accessory, or attached to an accessory or template polynucleotide, for bonding between a scaffold and a template polynucleotide or between a scaffold and an accessory, may be used for permitting or promoting bonding between such components as disclosed herein.

### Amplification

By way of brief example, following the attachment of the library DNA complexes to the DNA binding regions of the solid support (e.g., via hybridization of the accessory binding sites with the seeding primers), solid-phase amplification can then proceed. The first step of the amplification is a primer extension step in which nucleotides are added to the 3′ end of the immobilized clustering primer using the template to produce a fully extended complementary strand. The template is then typically washed off the solid support. The complementary strand will include at its 3′ end a clustering primer-binding sequence (i.e., either P5′ or P7′) which is capable of bridging to the second primer molecule immobilized on the solid support and binding. Further rounds of amplification (analogous to a standard PCR reaction) lead to the formation of clusters or colonies of template molecules bound to the solid support.

In some embodiments, a library DNA complex is captured as a double stranded template on the DNA binding region. Then immediately followed by invasion and strand displacement. It can be noted that the first strand synthesis step is not required since the library DNA template is already double stranded. After displacement, the original library template strand may bridge onto a complementary clustering primer. Also, the displaced strand can be extended. In some embodiments, when the accessory binding sites of a library DNA complex comprise an oligo sequence (e.g., PX′), such oligo sequence is not copied during clustering. The strands are thereafter extended via cluster amplification and sequenced.

### Solid Support

Some aspect of the present disclosure relates to a solid support for use in sequencing, having a patterned surface comprising a plurality of library DNA binding regions separated by interstitial regions;
wherein each library DNA binding region comprises a number of capture moieties adapted for capturing a library DNA complex, and at least a portion of the interstitial regions comprise clustering oligonucleotides; and
wherein the capture moieties are orthogonal to the clustering oligonucleotides, and the size, dimension or diameter of each library binding regions is from about 10 nm to about 100 nm, or about 20 nm to about 50 nm.

In some embodiments of the solid support described herein, the patterned surface comprises a plurality of nanowells, and at least a portion of the nanowells each comprises one library DNA binding region that is inside the nanowell. In some other embodiments, each of the library DNA binding region is a nanowell or a capture pad on the patterned surface.

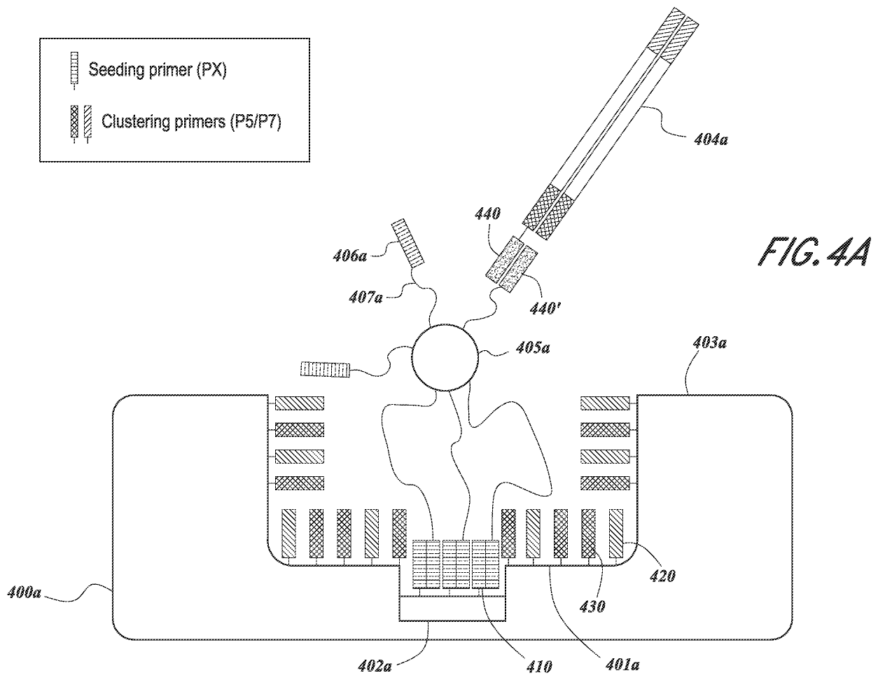

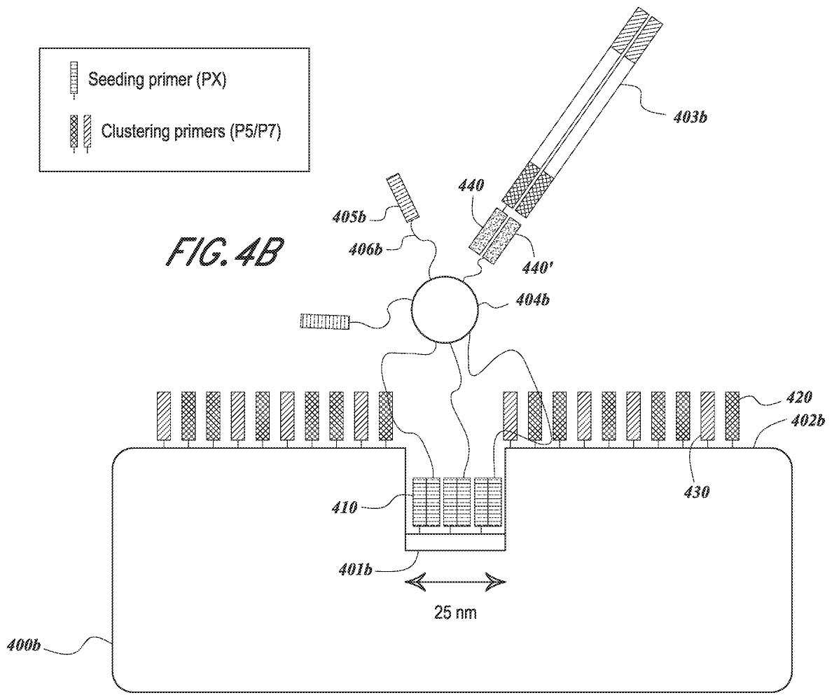

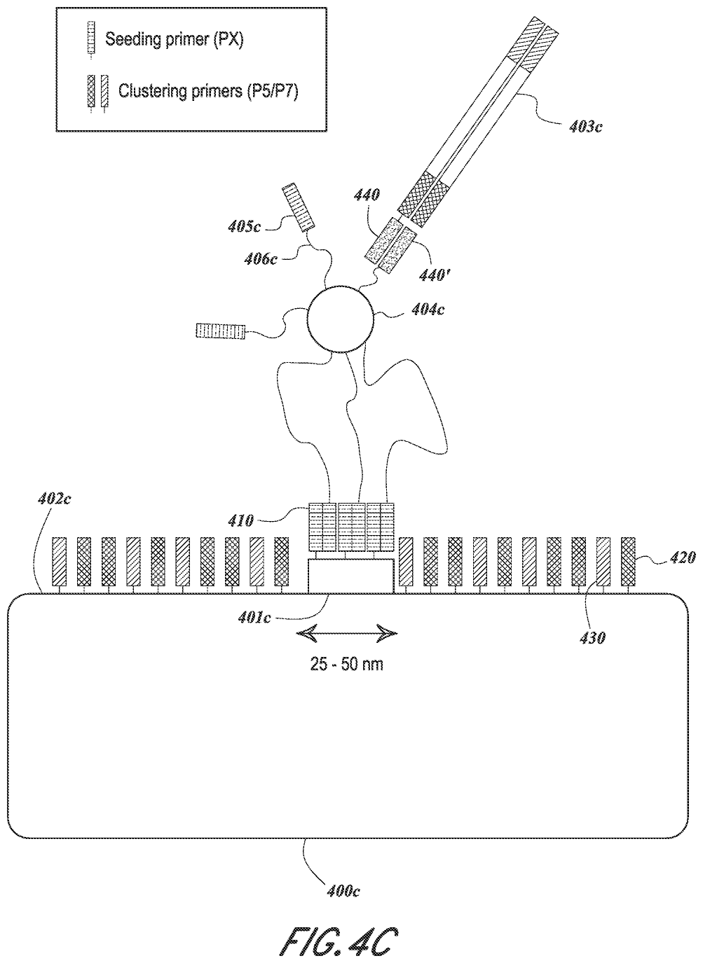

FIGS. 4A-4C each illustrates a cross-section view of a solid support according to an embodiment of the present disclosure. In each figure, the cross-section view shows a DNA binding region that is discrete or separated by the interstitial regions. In FIG. 4A, the DNA binding region comprising the surface-bound seeding primers 410 (i.e., capturing moieties) is within a smaller nanowell 402A located inside a bigger nanowell 401A grafted with clustering oligonucleotides 420 and 430. The interstitial region 403A does not contain either the seeding primers or the clustering primers. A library DNA complex 404A comprises a scaffold 405A with a number of accessory binding sites 406A. The library DNA complex 404A is attached to the DNA binding region by interaction of the seeding primers 410 with the accessory binding sites 406A, each comprising an oligo sequence that is complementary to seeding primer 410. There are more accessory binding sites 406A than the seeding primers 410. As such, all the seeding primers 410 are used in hybridization with the accessory binding sites 406A, excluding a second DNA complex from binding at the same DNA binding region. In FIG. 4B, the DNA binding region containing the seeding primers 410 is part of an array of ultra-small nanowell 401B that is discrete or separated by the interstitial regions 402B. The clustering oligonucleotides 420 and 430 are grafted on the interstitial regions 402B. Similar to FIG. 4A, a library DNA complex 403B comprising a scaffold 404B with a number of accessory binding sites 405B is attached to the DNA binding region by interaction of the seeding primers 410 with the accessory binding sites 405B, each comprising an oligo sequence that is complementary to seeding primer 410. In FIG. 4C, the DNA binding region containing the seeding primers 410 is on a capture pad 401C, and the clustering oligonucleotides 420 and 430 are in the interstitial regions 402C. A library DNA complex 403C comprising a scaffold 404C with a number of accessory binding sites 405C is attached to the DNA binding region by interaction of the seeding primers 410 with the accessory binding sites 405C, each comprising an oligo sequence that is complementary to seeding primer 410. For each of the library DNA complex in FIG. 4A-4C, the accessory binding sites may be attached to the scaffold 405A, 404B or 404C via an optional linker 407A, 407B or 406C respectively, or are parts of the scaffold. In further embodiments, the scaffold may be attached to the library polynucleotide (the insert and/or the adaptor sequence) by forming noncovalent or covalent interaction between a functionality 440 of the library polynucleotide and a complementary binding partner on the scaffold.

Some additional aspect of the present disclosure relates to a solid support for use in sequencing, having a patterned surface comprising a plurality of library DNA binding regions separated by interstitial regions;
wherein each library DNA binding region comprises a number of capture moieties adapted for capturing a library DNA complex, and at least a portion of the interstitial regions comprise clustering oligonucleotides;
wherein each library DNA complex comprises a single library polynucleotide and a number of accessory binding sites capable of binding to the capture moieties in the library DNA binding region by noncovalent or covalent interactions;
wherein the number of accessory binding sites on each library DNA complex is more than the number of capture moieties in each library DNA binding region; and
wherein the capture moieties are orthogonal to the clustering oligonucleotides.

In some embodiments of the solid support described herein, the solid support further comprises a plurality of library DNA complexes attached to the library binding regions through the capture moieties. In some such embodiments, at least 50%, 55%, 60%, 65%, 70%, 75%, 80%, 85%, 90%, 95%, 98%, or 99% of the library DNA binding regions are each occupied with no more than three library DNA complexes (e.g., one or two library DNA complexes). In further embodiments, at least 50%, 55%, 60%, 65%, 70%, 75%, 80%, 85%, 90%, 95%, 98%, or 99% of the library DNA binding regions are each occupied with a single library DNA complex or only a single dominant library DNA complex.

In some embodiments of the solid support described herein, each library DNA complex comprises a scaffold, wherein the number of accessory binding sites are on the scaffold, and the library polynucleotide is attached to the scaffold by non-covalent or covalent interaction with a single template binding site on the scaffold. In some such embodiments, the scaffold comprises a nanoparticle, a dendrimer, a polymer with bottlebrush structure, a single strand DNA scaffold, or a polypeptide scaffold, or a combination thereof. In further embodiments, the scaffold comprises a DNA dendrimer, a single strand DNA with bottlebrush structure, or a single strand DNA produced by rolling circle amplification (RCA). Nanoparticles solid support for library preparation have been described in U.S. Publication No. 2021/0187469 A1 and library DNA complex comprising a bottlebrush structure and the preparation of the same are described in U.S. Ser. No. 63/174,768, each of which is incorporated by reference in its entirety.

In some embodiments of the solid support described herein, the library polynucleotide is attached to the scaffold by noncovalent hybridization with the single template binding site. In other embodiments, the library polynucleotide is attached to the scaffold by covalent bonding with the single template binding site. In some such embodiments, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding. Additional disclosure on certain noncovalent and covalent interactions are described in detail in Table 1, as well as in the context of the library DNA complex.

In some embodiments of the solid support described herein, each capture moiety is adapted to capture the library DNA complex by non-covalent interaction with one or more accessory binding sites on the library DNA complex. In some such embodiments, each capture moiety comprises an oligonucleotide seeding sequence that is capable of hybridizing with one accessory binding site on the library DNA complex. In further embodiments, the oligonucleotide seeding sequence comprises from about 5 to about 50 nucleotides, from about 10 to about 40 nucleotides, or from about 20 to about 30 nucleotides. In other embodiments, each capture moiety is adapted to capture the library DNA complex by avidin (e.g., streptavidin) biotin interaction.

In some other embodiments of the solid support described herein, each capture moiety is adapted to capture the library DNA complex by forming a covalent bonding with one accessory binding site on the library DNA complex. In some such embodiments, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding. Additional disclosure on certain noncovalent and covalent interactions are described in detail in Table 1, as well as in the context of the library DNA complex.

In some embodiments of the solid support described herein, the solid support is a patterned flowcell, containing a plurality of nanowells on the patterned surface of the flowcell. In some further embodiments, at least a portion of the nanowells each comprises a single library DNA binding region that is inside the nanowell. In other embodiments, each library DNA binding region is a nanowell or a capture pad on the patterned surface of the flowcell. In some embodiments, the size, dimension or diameter of each library binding region is from about 10 nm to about 100 nm. In further embodiments, the size, dimension, or diameter of each library binding regions is about 10 nm, 20 nm, 30 nm, 40 nm, 50 nm, 60 nm, 70 nm, 80 nm, 90 nm, or 100 nm, or a range defined by any two of the preceding values. In further embodiments, the size, dimension, or diameter of each library binding regions is from about 20 nm to about 50 nm.

In some embodiments of the solid support described herein, each library DNA binding region comprises about 10, 20, 30, 40, 50, 60, 70, 80, 90, 100, 110, 120, 130, 140, 150, 160, 170, 180, 190 or 200 moieties, or a range defined by any of the two preceding values. In further embodiments, each library DNA binding region comprises from about from about 10 to about 100 capture moieties, or from about 20 to about 50 capture moieties. In some embodiments, the ratio of the clustering oligonucleotides to the capture moieties is from about 10 to 100,000. In further embodiments, the ratio of the clustering oligonucleotides to the capture moieties is about 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000, or 10,000, or a range defined by any two of the preceding values.

In some embodiments of the solid support described herein, the clustering oligonucleotides comprise P5 and P7 primers, or P15 and P17 primers.

In some embodiments, the substrate comprises patterned surfaces. For example, the substrate may make use of solid supports comprised of a substrate or matrix (e.g., glass slides) which has been “functionalized”, for example by application of a layer or coating of an intermediate material comprising reactive groups which permit covalent attachment to biomolecules, such as the clustering oligonucleotides and/or seeding primers. Examples of such supports include, but are not limited to, a substrate such as glass. In such embodiments, the biomolecules (e.g., clustering primers or seeding primers) may be directly covalently attached to the intermediate material but the intermediate material may itself be non-covalently attached to the substrate or matrix (e.g., the glass substrate). Alternatively, the substrate such as glass may be treated to permit direct covalent attachment of a biomolecule; for example, glass may be treated with hydrochloric acid, thus exposing the hydroxyl groups of the glass, and phosphite-triester chemistry used to directly attach a nucleotide to the glass via a covalent bond between the hydroxyl group of the glass and the phosphate group of the nucleotide. In one embodiment, the solid support may be functionalized with azido groups. In further embodiments, the azido groups may be introduced by an intermediate material such as a PAZAM coating.

In other embodiments, the solid support may be “functionalized” by application of a layer or coating of an intermediate material comprising groups that permit non-covalent attachment to biomolecules. In such embodiments, the groups on the solid support may form one or more of ionic bonds, hydrogen bonds, hydrophobic interactions, π-π interactions, van der Waals interactions and host-guest interactions, to a corresponding group on the biomolecules (e.g. polynucleotides). The interactions formed between the group on the solid support and the corresponding group on the biomolecules may be configured to cause immobilization or attachment under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing. In one embodiment, the solid support may be functionalized to introduce avidin bonding sites (e.g., streptavidin).

In other embodiments, the solid support may be “functionalized” by application of an intermediate material comprising groups that permit attachment via metal-coordination bonds to biomolecules. In such embodiments, the groups on the solid support may include ligands (e.g., metal-coordination groups), which are able to bind with a metal moiety on the biomolecule. Alternatively, or in addition, the groups on the solid support may include metal moieties, which are able to bind with a ligand on the biomolecule. The metal-coordination interactions formed between the ligand and the metal moiety may be configured to cause immobilization or attachment of the biomolecule under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

When referring to immobilization or attachment of molecules (e.g., nucleic acids) to a solid support, the terms “immobilized” and “attached” are used interchangeably herein and both terms are intended to encompass direct or indirect, covalent or non-covalent attachment, unless indicated otherwise, either explicitly or by context. In certain embodiments of the invention, covalent attachment may be preferred; in other embodiments, attachment using non-covalent interactions may be preferred; in yet other embodiments, attachment using metal-coordination bonds may be preferred. However, in general the molecules (e.g., nucleic acids) remain immobilized or attached to the support under the conditions in which it is intended to use the support, for example in applications requiring nucleic acid amplification and/or sequencing. When referring to attachment of nucleic acids to other nucleic acids, then the terms “immobilized” and “hybridized” are used herein, and generally refer to hydrogen bonding between complementary nucleic acids.

In some embodiments of the solid support described herein, the capture moieties of the DNA binding regions and/or the clustering oligonucleotides may be attached to the surface of the substrate through a polymer (including copolymer, may be random, block, linear, and/or branched copolymers) or a hydrogel, each of which comprising two or more recurring monomer units in any order or configuration, and may be linear, cross-linked, or branched, or a combination thereof. In an example, the polymer may be a heteropolymer and the heteropolymer may include an acrylamide monomer, such as

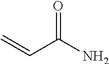

or a substituted analog thereof. In an example, the polymer is a heteropolymer and may further include an azido-containing acrylamide monomer. The polymer or hydrogel may be coated on the surface either by covalent or non-covalent attachment.

In some embodiments, the heteropolymer includes:

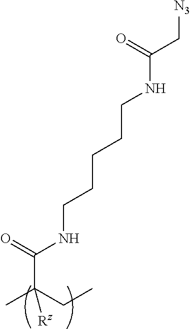

and optionally

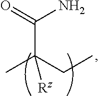

where each R^{z} is independently H or C₁₋₄ alkyl. In an example, a polymer used may include examples such as a poly(N-(5-azidoacetamidylpentyl) acrylamide-co-acrylamide), also known as PAZAM:

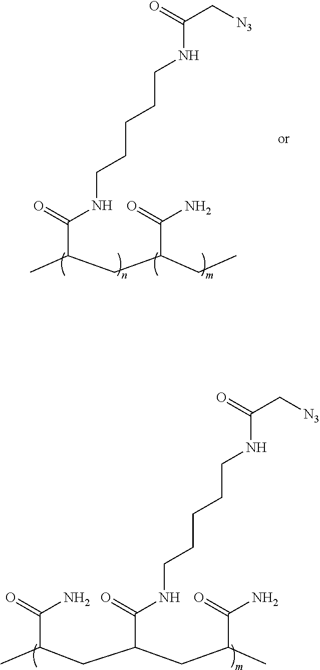

wherein n is an integer in the range of 1-20,000, and m is an integer in the range of 1-100,000. In some examples, the acrylamide monomer may include an azido acetamido pentyl acrylamide monomer:

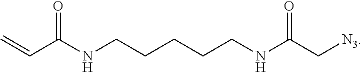 [SMILES: C=CC(=O)NCCCCCNC(=O)CN=[N+]=[N-]]

In some examples, the acrylamide monomer may include an N-isopropylacrylamide

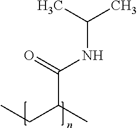

In some embodiments, the heteropolymer may include the structure:

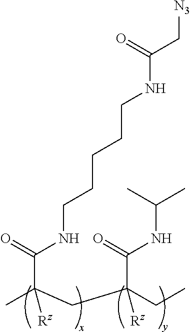

wherein x is an integer in the range of 1-20,000, and y is an integer in the range of 1-100,000, or

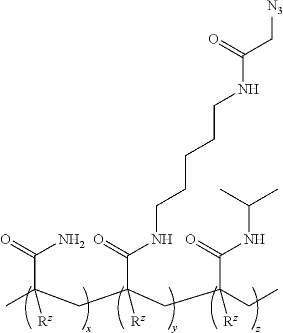

wherein y is an integer in the range of 1-20,000 and x and z are integers wherein the sum of x and z may be within a range of from 1 to 100,000, where each R^{z} is independently H or C₁₋₄ alkyl and a ratio of x:y may be from approximately 10:90 to approximately 1:99, or may be approximately 5:95, or a ratio of (x:y):z may be from approximately 85:15 to approximately 95:5, or may be approximately 90:10 (wherein a ratio of x:(y:z) may be from approximately 1:(99) to approximately 10:(90), or may be approximately 5:(95)), respectively.

### Sequencing Applications

Some embodiments are directed to methods of detecting an analyte using a substrate with a patterned surface prepared by the methods described herein. In some embodiments, the analyte is selected from nucleic acids, polynucleotides, proteins, antibodies, epitopes to antibodies, enzymes, cells, nuclei, cellular organelles, or small molecule drugs. In one embodiment, the analyte is a polynucleotide. In one embodiment, the detecting includes determining a nucleotide sequence of the polynucleotide.

Some embodiments that use nucleic acids can include a step of amplifying the nucleic acids on the substrate. Many different DNA amplification techniques can be used in conjunction with the substrates described herein. Exemplary techniques that can be used include, but are not limited to, polymerase chain reaction (PCR), rolling circle amplification (RCA), multiple displacement amplification (MDA), or random prime amplification (RPA). In particular embodiments, one or more oligonucleotide primers used for amplification can be attached to a substrate (e.g., via the azido silane layer). In PCR embodiments, one or both of the primers used for amplification can be attached to the substrate. Formats that utilize two species of attached primer are often referred to as bridge amplification because double stranded amplicons form a bridge-like structure between the two attached primers that flank the template sequence that has been copied. Exemplary reagents and conditions that can be used for bridge amplification are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420, each of which is incorporated herein by reference.

PCR amplification can also be carried out with one amplification primer attached to a substrate and a second primer in solution. An exemplary format that uses a combination of one attached primer and soluble primer is emulsion PCR as described, for example, in Dressman et al., Proc. Natl. Acad. Sci. USA 100:8817-8822 (2003), WO 05/010145, or U.S. Patent Publ. Nos. 2005/0130173 or 2005/0064460, each of which is incorporated herein by reference. Emulsion PCR is illustrative of the format and it will be understood that for purposes of the methods set forth herein the use of an emulsion is optional and indeed for several embodiments an emulsion is not used. Furthermore, primers need not be attached directly to substrate or solid supports as set forth in the ePCR references and can instead be attached to a gel or polymer coating as set forth herein.

RCA techniques can be modified for use in a method of the present disclosure. Exemplary components that can be used in an RCA reaction and principles by which RCA produces amplicons are described, for example, in Lizardi et al., Nat. Genet. 19:225-232 (1998) and US 2007/0099208 A1, each of which is incorporated herein by reference. Primers used for RCA can be in solution or attached to a gel or polymer coating.

MDA techniques can be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference. Primers used for MDA can be in solution or attached to a gel or polymer coating.

In particular embodiments, a combination of the above-exemplified amplification techniques can be used. For example, RCA and MDA can be used in a combination wherein RCA is used to generate a concatemeric amplicon in solution (e.g., using solution-phase primers). The amplicon can then be used as a template for MDA using primers that are attached to a substrate (e.g., via a gel or polymer coating). In this example, amplicons produced after the combined RCA and MDA steps will be attached to the substrate.

Substrates of the present disclosure that contain nucleic acid arrays can be used for any of a variety of purposes. A particularly desirable use for the nucleic acids is to serve as capture probes that hybridize to target nucleic acids having complementary sequences. The target nucleic acids once hybridized to the capture probes can be detected, for example, via a label recruited to the capture probe. Methods for detection of target nucleic acids via hybridization to capture probes are known in the art and include, for example, those described in U.S. Pat. Nos. 7,582,420; 6,890,741; 6,913,884 or 6,355,431 or U.S. Pat. Pub. Nos. 2005/0053980 A1; 2009/0186349 A1 or 2005/0181440 A1, each of which is incorporated herein by reference. For example, a label can be recruited to a capture probe by virtue of hybridization of the capture probe to a target probe that bears the label. In another example, a label can be recruited to a capture probe by hybridizing a target probe to the capture probe such that the capture probe can be extended by ligation to a labeled oligonucleotide (e.g., via ligase activity) or by addition of a labeled nucleotide (e.g., via polymerase activity).

In some embodiments, a substrate described herein can be used for determining a nucleotide sequence of a polynucleotide. In such embodiments, the method can comprise the steps of (a) contacting a substrate-attached polynucleotide/copy polynucleotide complex with one or more different type of nucleotides in the presence of a polymerase (e.g., DNA polymerase); (b) incorporating one type of nucleotide to the copy polynucleotide strand to form an extended copy polynucleotide; (c) perform one or more fluorescent measurements of one or more the extended copy polynucleotides; wherein steps (a) to (c) are repeated, thereby determining the sequence of the substrate-attached polynucleotide.

Nucleic acid sequencing can be used to determine a nucleotide sequence of a polynucleotide by various processes known in the art. In a preferred method, sequencing-by-synthesis (SBS) is utilized to determine a nucleotide sequence of a polynucleotide attached to a surface of a substrate (e.g., via any one of the polymer coatings described herein). In such a process, one or more nucleotides are provided to a template polynucleotide that is associated with a polynucleotide polymerase. The polynucleotide polymerase incorporates the one or more nucleotides into a newly synthesized nucleic acid strand that is complementary to the polynucleotide template. The synthesis is initiated from an oligonucleotide primer that is complementary to a portion of the template polynucleotide or to a portion of a universal or non-variable nucleic acid that is covalently bound at one end of the template polynucleotide. As nucleotides are incorporated against the template polynucleotide, a detectable signal is generated that allows for the determination of which nucleotide has been incorporated during each step of the sequencing process. In this way, the sequence of a nucleic acid complementary to at least a portion of the template polynucleotide can be generated, thereby permitting determination of the nucleotide sequence of at least a portion of the template polynucleotide.

Flow cells provide a convenient format for housing an array that is produced by the methods of the present disclosure and that is subjected to a sequencing-by-synthesis (SBS) or other detection technique that involves repeated delivery of reagents in cycles. For example, to initiate a first SBS cycle, one or more labeled nucleotides, DNA polymerase, etc., can be flowed into/through a flow cell that houses a nucleic acid array made by methods set forth herein. Those sites of an array where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with an array produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference in its entirety.

In some embodiments of the above-described method, which employ a flow cell, only a single type of nucleotide is present in the flow cell during a single flow step. In such embodiments, the nucleotide can be selected from the group consisting of dATP, dCTP, dGTP, dTTP, and analogs thereof. In other embodiments of the above-described method which employ a flow cell, a plurality different types of nucleotides are present in the flow cell during a single flow step. In such methods, the nucleotides can be selected from dATP, dCTP, dGTP, dTTP, and analogs thereof.

Determination of the nucleotide or nucleotides incorporated during each flow step for one or more of the polynucleotides attached to the polymer coating on the surface of the substrate present in the flow cell is achieved by detecting a signal produced at or near the polynucleotide template. In some embodiments of the above-described methods, the detectable signal comprises an optical signal. In other embodiments, the detectable signal comprises a non-optical signal. In such embodiments, the non-optical signal comprises a change in pH at or near one or more of the polynucleotide templates.

Applications and uses of substrates of the present disclosure have been exemplified herein with regard to nucleic acids. However, it will be understood that other analytes can be attached to a substrate set forth herein and analyzed. One or more analytes can be present in or on a substrate of the present disclosure. The substrates of the present disclosure are particularly useful for detection of analytes, or for carrying out synthetic reactions with analytes. Thus, any of a variety of analytes that are to be detected, characterized, modified, synthesized, or the like can be present in or on a substrate set forth herein. Exemplary analytes include, but are not limited to, nucleic acids (e.g., DNA, RNA or analogs thereof), proteins, polysaccharides, cells, antibodies, epitopes, receptors, ligands, enzymes (e.g., kinases, phosphatases or polymerases), small molecule drug candidates, or the like. A substrate can include multiple different species from a library of analytes. For example, the species can be different antibodies from an antibody library, nucleic acids having different sequences from a library of nucleic acids, proteins having different structure and/or function from a library of proteins, drug candidates from a combinatorial library of small molecules, etc.

In some embodiments, analytes can be distributed to features on a substrate such that they are individually resolvable. For example, a single molecule of each analyte can be present at each feature. Alternatively, analytes can be present as colonies or populations such that individual molecules are not necessarily resolved. The colonies or populations can be homogenous with respect to containing only a single species of analyte (albeit in multiple copies). Taking nucleic acids as an example, each feature on a substrate can include a colony or population of nucleic acids and every nucleic acid in the colony or population can have the same nucleotide sequence (either single stranded or double stranded). Such colonies can be created by cluster amplification or bridge amplification as set forth previously herein. Multiple repeats of a target sequence can be present in a single nucleic acid molecule, such as a concatemer created using a rolling circle amplification procedure. Thus, a feature on a substrate can contain multiple copies of a single species of an analyte. Alternatively, a colony or population of analytes that are at a feature can include two or more different species. For example, one or more wells on a substrate can each contain a mixed colony having two or more different nucleic acid species (i.e., nucleic acid molecules with different sequences). The two or more nucleic acid species in a mixed colony can be present in non-negligible amounts, for example, allowing more than one nucleic acid to be detected in the mixed colony.

What is claimed is:

1. A solid support for use in sequencing, having a patterned surface, comprising a plurality of library DNA binding regions separated by interstitial regions, and a plurality of library DNA complexes attached to the library DNA binding regions;
wherein each library DNA binding region comprises a number of capture moieties adapted for capturing a library DNA complex, and at least a portion of the interstitial regions comprise clustering oligonucleotides;
wherein each library DNA complex comprises a single library polynucleotide and a number of accessory binding sites capable of binding to the capture moieties in the library DNA binding region by noncovalent or covalent interactions;
wherein the number of accessory binding sites on each library DNA complex is more than the number of capture moieties in each library DNA binding region; and
wherein the plurality of library DNA complexes are attached to the library binding regions through the capture moieties, and wherein the capture moieties are orthogonal to the clustering oligonucleotides.

2. The solid support of claim 1, wherein at least 50% of the library DNA binding regions are each occupied with no more than three library DNA complexes.

3. The solid support of claim 1, wherein at least 50% of the library DNA binding regions are each occupied with only one library DNA complex or only one dominant library DNA complex.

4. The solid support of any one of claims 1 to 3, wherein each library DNA complex comprises a scaffold, wherein the number of accessory binding sites are on the scaffold, and the library polynucleotide is attached to the scaffold by non-covalent or covalent interaction with a single template binding site on the scaffold.

5. The solid support of claim 4, wherein the scaffold comprises a nanoparticle, a dendrimer, a polymer with bottlebrush structure, a single strand DNA scaffold, or a polypeptide scaffold.

6. The solid support of claim 4, wherein the scaffold comprises a DNA dendrimer, a single strand DNA with bottlebrush structure, or a single strand DNA produced by rolling circle amplification (RCA).

7. The solid support of any one of claims 4 to 6, wherein the library polynucleotide is attached to the scaffold by noncovalent hybridization with the single template binding site.

8. The solid support of any one of claims 4 to 6, wherein the library polynucleotide is attached to the scaffold by covalent bonding with the single template binding site.

9. The solid support of claim 8, wherein the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

10. The solid support of any one of claims 1 to 9, wherein at least a portion of the capture moieties are adapted to capture the library DNA complex by non-covalent interactions with one or more accessory binding sites on the library DNA complex.

11. The solid support of claim 10, wherein at least a portion of the capture moieties each comprises an oligonucleotide seeding sequence that is capable of hybridizing with one accessory binding site on the library DNA complex.

12. The solid support of claim 11, wherein the oligonucleotide seeding sequence comprises about 10 to about 40 nucleotides.

13. The solid support of claim 10, wherein at least a portion of the capture moieties are adapted to capture the library DNA complex by avidin biotin interaction.

14. The solid support of any one of claims 1 to 9, wherein at least a portion of the capture moieties are adapted to capture the library DNA complex by forming covalent bonding with accessory binding sites on the library DNA complex.

15. The solid support of any one of claims 1 to 14, wherein the solid support is a patterned flowcell, comprising a plurality of nanowells on the patterned surface of the flowcell.

16. The solid support of claim 15, wherein at least a portion of the nanowells each comprises a single library DNA binding region that is inside the nanowell.

17. The solid support of claim 15, wherein at least a portion of the library DNA binding regions are nanowells or capture pads, forming a patterned surface on the flowcell.

18. The solid support of any one of claims 1 to 17, wherein the size of each library binding regions is from about 10 nm to about 100 nm.

19. The solid support of any one of claims 1 to 18, wherein at least a portion of the library DNA binding regions each comprises from about 1 to 200 capture moieties.

20. The solid support of any one of claims 1 to 19, wherein the ratio of the clustering oligonucleotides to the capture moieties is from about 10 to 100,000.

21. The solid support of any one of claims 1 to 20, wherein the clustering oligonucleotides comprise P5 and P7 primers, or P15 and P17 primers.

22. A method of preparing a patterned surface of a solid support for sequencing, comprising:
contacting a buffer solution comprising a plurality of library DNA complexes with the patterned surface comprising a plurality of library DNA binding regions separated by interstitial regions;
wherein each library DNA binding region comprises a number of capture moieties adapted for capturing one library DNA complex, at least a portion of the interstitial regions comprise clustering oligonucleotides, and the capture moieties are orthogonal to the clustering oligonucleotides;
wherein each library DNA complex comprises a single library polynucleotide and a number of accessory binding sites, and the number of accessory binding sites on each library DNA complex is more than the number of capture moieties in each library DNA binding region; and
attaching the plurality of library DNA complexes to the library DNA binding regions of the pattered surface by noncovalent or covalent interactions between the accessory binding sites of the library DNA complexes and the capture moieties of library DNA binding regions.

23. The method of claim 22, wherein at least 50% of the library DNA binding regions are each occupied with no more than three library DNA complexes.

24. The method of claim 22 or 23, wherein at least 50% of the library DNA binding regions are each occupied with only one library DNA complex or only one dominant library DNA complex.

25. The method of any one of claims 22 to 24, further comprising amplifying the library polynucleotides.

26. The method of claim 25, wherein at least 50% of clusters generated from the amplification are monoclonal clusters.

27. The method of any one of claims 22 to 26, wherein the solid support comprises a flowcell.

28. The method of claim 27, wherein the flowcell has a patterned surface comprising a plurality of nanowells, and at least a portion of the nanowells each comprises a single library DNA binding region that is inside the nanowell.

29. The method of claim 27, wherein each of the library DNA binding region is a nanowell or a capture pad, forming a patterned surface on the flowcell.

30. The method of any one of claims 22 to 29, wherein the size of each library binding regions is from about 10 nm to about 100 nm, or about 20 nm to about 50 nm.

31. The method of any one of claims 22 to 30, wherein each library DNA complex comprises a scaffold, wherein the number of accessory binding sites are on the scaffold, and the single library polynucleotide is attached to the scaffold by non-covalent or covalent interaction with a single template binding site on the scaffold.

32. The method of claim 31, wherein the scaffold comprises a nanoparticle, a dendrimer, a polymer with bottlebrush structure, a single strand DNA scaffold, or a polypeptide scaffold.

33. The method of claim 31 or 32, wherein the library polynucleotide is attached to the scaffold by noncovalent hybridization with the single template binding site, or by covalent bonding with the single template binding site.

34. The method of any one of claims 22 to 33, wherein at least a portion of the capture moieties each comprises an oligonucleotide seeding sequence, and each library DNA complex is attached to the library DNA binding region by hybridization of one or more accessory binding sites on the library DNA complex with the oligonucleotide seeding sequences.

35. The method of claim 34, wherein the oligonucleotide seeding sequence comprises about 10 to about 40 nucleotides.

36. The method of any one of claims 22 to 33, wherein at least a portion of the capture moieties each comprises an avidin moiety, at least a portion of the accessory binding sites on the library DNA complex each comprises a biotin moiety, and the library DNA complex is attached to the library DNA binding region by avidin biotin interaction.

37. The method of any one of claims 22 to 33, wherein each library DNA complex is attached to the library DNA binding region by covalent bonding selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

38. The method of any one of claims 22 to 37, wherein at least a portion of the library DNA binding region each comprises from about 1 to 200 capture moieties.

39. The method of any one of claims 22 to 38, wherein the ratio of the clustering oligonucleotides to the capture moieties is from about 10 to 100,000.

40. The method of any one of claims 22 to 39, wherein the clustering oligonucleotides comprise P5 and P7 primers, or P15 and P17 primers.

41. A DNA library comprising a plurality of library DNA complexes, each library DNA complex comprises a single library polynucleotide, and a scaffold comprising a number of accessory binding sites adapted for attaching to a number of capture moieties on a library DNA binding region of a patterned solid support,
wherein the library polynucleotide comprises an insert and an adaptor region;
wherein the adaptor region comprises a clustering sequence, each accessory binding sites comprises a complementary capture moiety, wherein the clustering sequence and the complementary capture moiety are orthogonal; and
wherein the number of accessory binding sites on each library DNA complex is more than the number of capture moieties in the library DNA binding region.

42. The DNA library of claim 41, wherein the library DNA complex further comprises a spacer region between the clustering sequence and the scaffold.

43. The DNA library of claim 41 or 42, wherein the spacer region comprises a linker.

44. The DNA library of claim 43, wherein the linker comprises a PEG linker.

45. The DNA library of any one of claims 41 to 44, wherein the library polynucleotide further comprises an index sequence, a first sequencing binding site, a second sequencing binding site, and/or a second index sequence.

46. The DNA library of any one of claims 41 to 45, wherein the adaptor region comprises a P5, P7, P15 or P17 sequence, or a sequence that is complementary to P5, P7, P15 or P17 sequence.

47. The DNA library of any one of claims 41 to 46, wherein the library is a double-stranded library.

48. The DNA library of any one of claims 41 to 47, wherein the scaffold comprises a DNA dendrimer, a polymer with bottlebrush structure, a single strand DNA with bottlebrush structure, a single strand DNA produced by rolling circle amplification, or a polypeptide scaffold, or a combination thereof.

49. The DNA library of any one of claims 41 to 48, wherein each complementary capture moiety of the library DNA complex comprises an oligo sequence that is hybridizable to the capture moiety on the library binding region of the patterned solid support.


SEQUENCE LISTING (16 sequences)

SEQ ID NO: 1 - DNA, 29 bp, synthetic construct
  misc_feature 1..29: note = P5 primer
  source 1..29: mol_type = other DNA
  misc_feature 23: note = n is a deoxyuridine
aatgatacggcgaccaccgaganctacac

SEQ ID NO: 2 - DNA, 24 bp, synthetic construct
  misc_feature 1..24: note = P7 primer
  source 1..24: mol_type = other DNA
caagcagaagacggcatacgagat

SEQ ID NO: 3 - DNA, 20 bp, synthetic construct
  misc_feature 1..20: note = P5 primer
  source 1..20: mol_type = other DNA
aatgatacggcgaccaccga

SEQ ID NO: 4 - DNA, 21 bp, synthetic construct
  misc_feature 1..21: note = P7 primer
  source 1..21: mol_type = other DNA
caagcagaagacggcatacga

SEQ ID NO: 5 - DNA, 29 bp, synthetic construct
  misc_feature 1..29: note = P15 primer
  misc_feature 23: note = T is an allyl modified T
  source 1..29: mol_type = other DNA
aatgatacggcgaccaccgagatctacac

SEQ ID NO: 6 - DNA, 27 bp, synthetic construct
  misc_feature 1..27: note = P17 primer
  misc_feature 1..3: note = Each n is a diol linker subject to chemical cleavage
  source 1..27: mol_type = other DNA
nnncaagcagaagacggcatacgagat

SEQ ID NO: 7 - DNA, 24 bp, synthetic construct
  misc_feature 1..24: note = PX'
  source 1..24: mol_type = other DNA
cctcctcctcctcctcctcctcct

SEQ ID NO: 8 - DNA, 24 bp, synthetic construct
  misc_feature 1..24: note = PX
  source 1..24: mol_type = other DNA
aggaggaggaggaggaggaggagg

SEQ ID NO: 9 - DNA, 35 bp, synthetic construct
  misc_feature 1..35: note = PA
  source 1..35: mol_type = other DNA
gctggcacgtccgaacgcttcgttaatccgttgag

SEQ ID NO: 10 - DNA, 35 bp, synthetic construct
  misc_feature 1..35: note = cPA (PA'),
  source 1..35: mol_type = other DNA
ctcaacggattaacgaagcgttcggacgtgccagc

SEQ ID NO: 11 - DNA, 35 bp, synthetic construct
  misc_feature 1..35: note = PB,
  source 1..35: mol_type = other DNA
cgtcgtctgccatggcgcttcggtggatatgaact

SEQ ID NO: 12 - DNA, 35 bp, synthetic construct
  misc_feature 1..35: note = cPB (PB')
  source 1..35: mol_type = other DNA
agttcatatccaccgaagcgccatggcagacgacg

SEQ ID NO: 13 - DNA, 35 bp, synthetic construct
  misc_feature 1..35: note = PC,
  source 1..35: mol_type = other DNA
acggccgctaatatcaacgcgtcgaatccgcaact

SEQ ID NO: 14 - DNA, 35 bp, synthetic construct
  misc_feature 1..35: note = cPC (PC'),
  source 1..35: mol_type = other DNA
agttgcggattcgacgcgttgatattagcggccgt

SEQ ID NO: 15 - DNA, 34 bp, synthetic construct
  misc_feature 1..34: note = PD
  source 1..34: mol_type = other DNA
gccgcgttacgttagccggactattcgatgcagc

SEQ ID NO: 16 - DNA, 34 bp, synthetic construct
  misc_feature 1..34: note = cPD (PD'),
  source 1..34: mol_type = other DNA
gctgcatcgaatagtccggctaacgtaacgcggc